SEQUENCE LISTING (2 sequences)

SEQ ID NO: 1 - protein, 406 aa, Artificial Sequence
  ANGPTL4 SEQUENCE
MSGAPTAGAALMLCAATAVLLSAQGGPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 2 - protein, 283 aa, Artificial Sequence
  ANGPTL4 SEQUENCE
  misc_feature (39)..(39): Xaa can be any naturally occurring amino acid
MSGAPTAGAALMLCAATAVLLSAQGGPVQSKSPRFASWXAANPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

(19) United States
(12) Patent Application Publication
(10) Pub. No.: US 2017/0354714 A1
(43) Pub. Date: Dec. 14, 2017

(54) ANTI-OBESITY AND ANTI-DIABETIC EFFECTS OF ANGIOPOIETIN-LIKE 4 (ANGPTL4) FIBRINOGEN-LIKE DOMAIN

(71) Applicant: THE REGENTS OF THE UNIVERSITY OF CALIFORINA, Oakland, CA (US)

(72) Inventor: Jen-Chywan WANG, San Francisco, CA (US)

(21) Appl. No.: 15/529,185

(22) PCT Filed: Nov. 25, 2015
(86) PCT No.: PCT/US15/62718

§ 371 Date: May 24, 2017

Related U.S. Application Data
(60) Provisional application No. 62/084,392, filed on Nov. 25, 2014.

(51) Int. Cl. A61K 38/18; C07K 14/515

(52) U.S. Cl. CPC A61K 38/1891; C07K 14/515

(57) ABSTRACT

Angiopoietin-like 4 (Angpt14) is a secreted protein that inhibits lipoprotein lipase (LPL) activity and promotes lipolysis in adipocytes. The present methods provides compositions comprising ANGPTL4 FLD and variants thereof of methods of using such compositions to protect a subject diet-induced obesity without affecting plasma TG levels, and, in an exemplary emobiment improve glucose homeostasis. The invention provides compositions comprising Angpt14 FLD and variants thereof and methods of using these compositions as a therapeutic agent against metabolic diseases, such as obesity and type 2 diabetes.

### CROSS REFERENCE TO RELATED APPLICATIONS

The present application claims priority under 35 U.S.C. §119(e) to U.S. Provisional Patent Application No. 62/084,392 filed Nov. 25, 2014, which is incorporated herein by reference in its entirety.

### SPONSORED RESEARCH OR DEVELOPMENT

This invention was made with Government support under Grant No. R01 DK083591 awarded by NIH. The Government has certain rights in the invention.

### BACKGROUND OF THE INVENTION

Diabetes mellitus, or diabetes, is a chronic disease that is characterized by impaired glucose regulation. Diabetes can be divided into two clinical syndromes, Type 1 diabetes mellitus and Type 2 diabetes mellitus (T2D). In type 1 diabetes, previously called juvenile-onset or insulin-dependent, insulin production is absent due to autoimmune pancreatic (3-cell destruction. Although the pathogenesis of autoimmune 3-cell destruction is not completely understood, it is believed to involve interactions between susceptibility genes, autoantigens, and environmental factors. Type 1 diabetes generally develops in childhood or adolescence and accounts for about 10% of all cases of diabetes.

In Type 2 diabetes, previously called adult-onset or non-insulin-dependent, insulin production may or may not be inadequate, but the body is unable to utilize the insulin that is present to normalize glucose levels in the body. It is caused by a combination of poorly understood genetic and acquired risk factors, including high-fat diet, lack of exercise, and aging. Type 2 diabetes accounts for about 90% of the cases of diabetes around the world, and is estimated to affect more than 220 million people worldwide. Although it more commonly occurs in adults, Type 2 diabetes is now becoming more common in children.

Chronic diabetes can lead to long-term complications affecting various organs, especially the heart, blood vessels, eyes, kidneys, and nerves. Signs of severe brain dysfunction often occur in subjects with obesity and insulin resistance/hyperinsulinemia. To date, however, there is no brain disease diagnostic method or treatment specific to diabetics.

The prevalence of obesity-related metabolic syndrome consisting of diabetes, hypertension, hypertriglyceridemia, hepatic steatosis, and accelerated atherosclerosis is increasing worldwide. No satisfactory treatments are available for obesity and the metabolic syndrome. Thus, there continues to be a need in the art for effective treatments for diabetes, obesity and metabolic disorders. The present invention provides such treatments.

### BRIEF SUMMARY OF THE INVENTION

Obesity and its associated metabolic diseases, including type 2 diabetes, are components of a global epidemic. However, the pharmacological approaches against obesity and metabolic disease are limited.

Currently anti-obesity drugs approved by FDA mainly suppress appetite or fat absorption in gut. These drugs can cause adverse effects, such as depression and steatorrhea. In an exemplary embodiment, the present invention directly targets both white and brown adipose tissue, thus it provides a different approach to reduce obesity, complementing current pharmacological therapies. In addition, the invention provides compositions and methods to improve insulin sensitivity in type 2 diabetes patients.

Angiopoietin-like 4 (Angptl4) is a secreted protein that inhibits lipoprotein lipase (LPL) activity and promotes lipolysis in adipocytes. Previous studies have shown that the N-terminal coiled coil domain of Angptl4 alone can inhibit LPL. In contrast, the present invention is at least partially based on the discovery that the C-terminal fibrinogen-like region is active in combating obesity and metabolic disease.

In various embodiments, the present invention provides compositions comprising the C-terminal fibrinogen-like domain (FLD) of Angptl4, and variants thereof, which have been discovered to elevate intracellular cAMP levels and induce lipolysis in adipocytes.

In various embodiments, the present invention provides novel compositions and methods for preventing and reducing obesity, and improving insulin sensitivity of obese and type 2 diabetes patients.

In various embodiments, the invention provides compositions and methods for increasing Angptl4-FLD, or a variant thereof, in circulation in a subject, thereby reducing adiposity without elevating plasma TG levels.

In an exemplary embodiment, the invention provides compositions and methods for increasing Angptl4-FLD, or a variant thereof, in circulation in a subject, thereby improving glucose homeostasis.

At least in part, the present invention is based on the discovery that Angptl4, and variants thereof comprising at least a subsequence of the FLD domain of Angptl4, curtail the development of obesity, insulin resistance, diabetes, hypertriglyceridemia, and hepatic steatosis, and increases energy expenditure.

Thus, in an exemplary embodiment, the invention provides isolated polypeptides comprising a variant of the naturally occuring sequence of Angptl4, comprising at least a subsequence of the FLD domain of Angptl4. An exemplary naturally occuring Angplt sequence is:

SEQ. ID. NO.: 1
MSGAPTAGAA LMLCAATAVL LSAQGGPVQS KSPRFASWDE
MNVLAHGLLQ LGQGLREHAE RTRSQLSALE RRLSACGSAC
QGTEGSTDLP LAPESRVDPE VLHSLQTQLK AQNSRIQQLF
HKVAQQQRHL EKQHLRIQHL QSQFGLLDHK HLDHEVAKPA
RRKRLPEMAQ PVDPAHNVSR LHRLPRDCQE LFQVGERQSG
LFEIQPQGSP PFLVNCKMTS DGGWTVIQRR HDGSVDFNRP
WEAYKAGFGD PHGEFWLGLE KVHSITGDRN SRLAVQLRDW
DGNAELLQFS VHLGGEDTAY SLQLTAPVAG QLGATTVPPS
GLSVPFSTWD QDHDLRRDKN CAKSLSGGWW FGTCSHSNLN
GQYFRSIPQQ RQKLKKGIFW KTWRGRYYPL QATTMLIQPM AAEAAS.

In some embodiments, the invention provides an isolated variant of SEQ. ID. NO.: 1 in which at least amino acids 38-165 are deleted.

In various embodiments, the polypeptide of the invention does not include a signal peptide sequence.

In various embodiments, the polypeptide of the invention includes amino acids 165-406 of SEQ. ID. NO.: 1. In various embodiments, the invention provides an isolated polypeptide having a sequence which has at least about 90%, at least about 91%, at least about 92%, at least about 93%, at least about 94%, at least about 95%, at least about 96%, at least about 97%, at least about 98% or at least about 99% sequence identity with a polypeptide including amino acids 165-406 of SEQ. ID. NO.: 1. In various embodiments, the invention provides a polypeptide in which the sequence of amino acids 165-406 of SEQ. ID. NO.: 1 is the only sequence in the polypeptide corresponding to SEQ. ID. NO.: 1.

In various embodiments, the polypeptide of the invention comprises amino acids 184-406 of SEQ. ID. NO.: 1. In various embodiments, the invention provides an isolated polypeptide having a sequence which has at least about 90%, at least about 91%, at least about 92%, at least about 93%, at least about 94%, at least about 95%, at least about 96%, at least about 97%, at least about 98% or at least about 99% sequence identity with a polypeptide including amino acids 184-406 of SEQ. ID. NO.: 1. In various embodiments, the invention provides a polypeptide in which the sequence of amino acids 184-406 of SEQ. ID. NO.: 1 is the only sequence in the polypeptide corresponding to SEQ. ID. NO.: 1.

In various embodiments, the invention provides an isolated polypeptide having a sequence which has at least about 90%, at least about 91%, at least about 92%, at least about 93%, at least about 94%, at least about 95%, at least about 96%, at least about 97%, at least about 98% or at least about 99% sequence identity with a polypeptide of SEQ. ID. NO.: 2:

SEQ. ID. NO.: 2
MSGAPTAGAA LMLCAATAVL LSAQGGPVQS KSPRFASW(Xaa)ₙ
PEMAQ PVDPAHNVSR LHRLPRDCQE LFQVGERQSG LFEIQPQGSP
PFLVNCKMTS DGGWTVIQRR HDGSVDFNRP WEAYKAGFGD
PHGEFWLGLE KVHSITGDRN SRLAVQLRDW DGNAELLQFS
VHLGGEDTAY SLQLTAPVAG QLGATTVPPS GLSVPFSTWD
QDHDLRRDKN CAKSLSGGWW FGTCSHSNLN GQYFRSIPQQ
RQKLKKGIFW KTWRGRYYPL QATTMLIQPM AAEAAS.

In SEQ. ID. NO.: 2, Xaa is a naturally occurring amino acid or is an uncoded amino acid. The index n includes the integers 0, 1, 2, 3, 4, 5, 6, 7, 8, 9, 10 or higher. In an exemplary embodiment, (Xaa)ₙ is a linker. In an exemplary embodiment, (Xaa)ₙ is an amino acid that provides a locus for conjugation of a heterologous moiety to the reamainder of the polypeptide according to SEQ. ID. NO.: 2, or (Xaa)ₙ represents that amino acid and heterologous moiety conjugate.

In various embodiments, the invention provides fusion peptides including a peptide of the invention fused to a second peptide, e.g., SEQ. ID. NO. 2 fused to a cell-penetrating peptide. In some embodiments, the cell-penetrating peptide is fused on the C-terminus of the peptide. In some embodiments, the cell-penetrating peptide is fused on the N-terminus of the peptide. In some embodiments, the cell-penetrating peptide is selected from the group consisting of HIV-derived TAT peptide, penetratins, transportans, SS peptides, and hCT derived cell-penetrating peptides.

In various embodiments, the isolated polypeptides or fusion peptides, upon administration to a subject, act to protect the subject from obesity, reduce adiposity and the like. In an exemplary embodiment, the isolated polypeptide or fusion peptide, upon administration to a subject, acts to protect the subject from obesity, reduce adiposity and the like without significant effect on the plasma triglyceride levels of the subject.

In yet another aspect, the invention provides isolated nucleic acids encoding the polypeptides or fusion peptides described herein, and host cells including and/or expressing the isolated nucleic acids.

In an additional aspect, the invention provides therapeutic compositions including the isolated polypeptides described herein in a physiologically acceptable carrier. In some embodiments, the compositions further include at least one cell-penetrating agent, e.g., a cationic liposome.

Also provided herein is the use of the isolated polypeptides or fusion peptides described herein in the treatment of obesity or an obesity-related disorder. In some embodiments, the obesity-related disorder is diabetes or metabolic syndrome, hepatic steatosis, non-hepatic steatosis or hypertriglyceridemia.

In yet another aspect, the invention features methods for treating obesity or an obesity-related disorder in a subject. The methods include administering a therapeutically effective amount of a peptide or fusion peptide of the invention to a subject in need of such treatment. In some embodiments, the obesity-related disorder is diabetes or metabolic syndrome, hepatic steatosis, non-hepatic steatosis or hypertriglyceridemia.

Additional embodiments, objects and advantages of the present invention are apparent from the detailed description set forth below.

### BRIEF DESCRIPTION OF THE DRAWINGS

FIG. 1. Proposed model of FLD action in mice. Angiopoietin-like 4 fibrinogen-like domain (ANGPTL4-FLD) improves glucose homeostasis. ANGPTL4-FLD improves glucose homeostasis by several potential mechanisms. First, elevating circulating ANGPTL4-FLD reduces hepatic gluconeogenesis that decreases plasma glucose levels. Second, ANGPTL4-FLD promotes lipolysis in white adipose tissues (WAT), which reduces adiposity that is associated with improved glucose homeostasis. Moreover, ANGPTL4-FLD induces “browning” of WAT that increases energy expenditure. ANGPTL4-FLD also increases lipolysis and uncoupling protein-1 (UCP-1) expression in brown adipose tissues (BAT) that potentiates energy expenditure. Increasing energy expenditure can improve insulin sensitivity.

FIG. 2A and FIG. 2B. Angptl4(WT), FLD and E40K increased A) glycerol release, B) intracellular cAMP in mouse primary adipocytes. N=4 +/−SEM,*P<0.05.

FIG. 3. The expression of Angptl4and FLD (red arrow) in plasma of mice infected with adenovirus expressing LacZ, Angptl4, and FLD for 3 weeks.

FIG. 4A and FIG. 4B. Mice expressing ANGPTL4 and FLD gained less weight than mice expressing LacZ under high-fat diet, despite similar food intake. N=5-6+/−SEM, *p<0.05.

FIG. 5. The weight of different tissues in mice overexpressing LacZ, Angptl4WT, AngpltFLD under high fat diet (n=5-6). *p<0.05.

FIG. 6. eWAT and iWAT in mice overexpressing LacZ, Angptl4, and FLD under high-fat diet. Tissues are from a representative mouse.

FIG. 7. Plasma TG levels of mice expressing LacZ, FLD, and Angptl4. N=5-6, *p<0.05 (vs LacZ).

FIG. 8. FLD-overexpressing mice had improved glucose tolerance than LacZ and ANGPTL4-overexpressing mice. Mice were under high fat diet. N=5-6, *p<0.05.

FIG. 9A and FIG. 9B. TG levels in A) liver and B) gastrocnemius muscle were lower in FLD- and ANGPTL4-overexpressing mice than those of WT mice. N=5-6, *p<0.05.

FIG. 10. Relative expression of fatty acid oxidative gene expression in LacZ, ANGPTL4 and FLD-overexpressing mice. N=3-5, *p<0.05 (vs. LacZ mice).

FIG. 11. FLD- and ANGPTL4-overexpressing mice had improved glucose tolerance than LacZ-overexpressing mice. Mice were under high fat diet. N=5-6, *p<0.05 (FLD vs. LacZ).

FIG. 12A and FIG. 12B. A) PPT for LacZ- and FLD-expressing mice under chow diet. N=5-6, B) Hepatic Pepck gene expression in these mice. *p<0.05 (FLD vs. LacZ).

FIG. 13A and FIG. 13B. Mice overexpressing Angptl4WT and Angptl4FLD had less weight gain than mice overexpressing LacZ under high fat diet, but their food intake was similar. N=5-6 +/−SEM, *p<0.05.

FIG. 14A and FIG. 14B. Mice overexpressing Angptl4WT and Angptl4FLD showed no change in weight gain and had similar food intake compared to mice overexpressing LacZ on chow diet. N=5-6 +/−SEM, *p<0.05.

FIG. 15. Overexpression of Angptl4FLD significantly reduced the expression of gluconeogenic genes PEPCK and G6Pase (n=5-6). *p<0.05.

FIG. 16. VO2 assayed by indirect calorimetry in lacZ (control) and FLD-overexpressing mice on high fat diet (n=6 mice per group).

FIG. 17. VCO2 (left) and RER (right) assayed by indirect calorimetry in lacZ (control) and FLD-overexpressing mice on high fat diet (n=6 mice per group).

FIG. 18. VO2 assayed by indirect calorimetry in lacZ (control) and FLD-overexpressing mice on chow diet (n=3 mice per group).

FIG. 19. VCO2 assayed by indirect calorimetry in lacZ (control) and FLD-overexpressing mice on chow diet (n=3 mice per group).

FIG. 20. RT-qPCR for selected liver fatty acid oxidation genes from liver of lacZ of FLD overexpressing mice.

FIG. 21. Plasma FGF21 levels of lacZ of FLD overexpressing mice.

### DETAILED DESCRIPTION OF THE INVENTION

### I. Introduction

Angiopoeitin-like protein 4 is conserved among several mammalian species. Ge et al. (2004) J. Biol. Chem. 279:2038-2045. Angiopoeitin-like protein 4 contains an N-terminal coiled-coil domain and a C-terminal fibrinogen-like domain. Kim et al. (2000) Biochem. J. 346:603-610. The N-terminal coiled-coil domain mediates oligomerization of angiopoeitin-like protein 4. Ge et al. (2004) J. Biol. Chem. 279:2038-2045. Oligomerized angiopoeitin-like protein 4 undergoes proteolytic processing in vivo, resulting in the cleavage of the fibrinogen-like domain. Ge et al. (2004) J. Biol. Chem. 279:2038-2045.

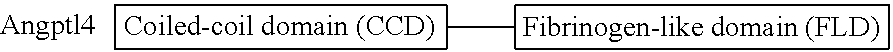

Angptl4 is a secreted protein that consists of an N-terminal coiled coil domain (CCD) and a C-terminal fibrinogen-like domain (FLD). Overexpression of Angptl4 causes hyperlipidemia. CCD alone can inhibit lipoprotein lipase (LPL). Angptl4 expression is induced by glucocorticoids, fasting, hypoxia and stress. Angptl4 is also a target gene of PPARα, β/δ and PPARγ. Angptl4 can induce lipolysis in adipocytes via activation of the cAMP-PKA signaling pathway.

Angiopoietin-like 4 (Angptl4) is secreted from adipose tissue and liver that acts in an endocrine, paracrine and/or autocrine fashion. Angptl4 exists either in full-length, or truncated forms, as N-terminal coiled-coil domain (CCD) or C-terminal fibrinogen-like domain (FLD) in plasma or tissues. The inventors discovered, inter alia, that FLD increases intracellular cAMP levels to promote lipolysis, a process in which triglycerides (TG) are hydrolyzed to fatty acids and glycerol, in both white and brown adipocytes.

### I. Definitions

Before the invention is described in greater detail, it is to be understood that the invention is not limited to particular embodiments described herein as such embodiments may vary. It is also to be understood that the terminology used herein is for the purpose of describing particular embodiments only, and the terminology is not intended to be limiting. The scope of the invention will be limited only by the appended claims. Unless defined otherwise, all technical and scientific terms used herein have the same meaning as commonly understood by one of ordinary skill in the art to which this invention belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the invention described herein is not entitled to antedate such publication by virtue of prior invention. Further, the dates of publication provided might be different from the actual publication dates, which may need to be independently confirmed.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features which may be readily separated from or combined with the features of any of the other several embodiments without departing from the scope or spirit of the invention. Any recited method may be carried out in the order of events recited or in any other order that is logically possible. Although any methods and materials similar or equivalent to those described herein may also be used in the practice or testing of the invention, representative illustrative methods and materials are now described.

In describing the present invention, the following terms will be employed, and are defined as indicated below.

The practice of the present invention will employ, unless otherwise indicated, conventional techniques of molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds), Immunochemical Methods In Cell And Molecular Biology (Mayer and Walker, eds., Academic Press, London, 1987); and Handbook Of Experimental Immunology, Volumes I-IV (D. M. Weir and C. C. Blackwell, eds., 1986).

The term “Angptl4” refers to an angiopoietin like protein 4 from any vertebrate or mammalian source, including, but not limited to, human, bovine, chicken, rodent, mouse, rat, porcine, ovine, primate, monkey, and guinea pig, unless specified otherwise. The term also refers to fragments and variants of native Angptl4that maintain at least one in vivo or in vitro activity of a native ANGPTL4. The term encompasses full-length unprocessed precursor forms of Angptl4as well as mature forms resulting from post-translational cleavage of the signal peptide and forms resulting from proteolytic processing of the fibrinogen domain. In certain embodiments, a full-length, unprocessed mouse Angptl4has the amino acid sequence set forth in SEQ ID NO: 1.

The term “Angplt4-FLD” refers to the fibrinogen-like domain of Angptl4, and variants thereof.

The term “subject” includes human and animal subjects. In certain embodiments, a subject is a mammal. In certain such embodiments, a subject is a human.

A “fragment” of a polypeptide refers to a contiguous stretch of amino acids from any portion of the Angplt-FLD polypeptide. A fragment may be of any length that is less than the length of the reference polypeptide.

A “variant” of a Angplt4 or Angplt-FLD polypeptide refers to a polypeptide having one or more amino acid substitutions, deletions, or insertions relative to the reference polypeptide.

A “conservative” amino acid substitution refers to the substitution of an amino acid in a polypeptide with another amino acid having similar properties, such as size or charge. In certain embodiments, a polypeptide comprising a conservative amino acid substitution maintains at least one activity of the unsubstituted polypeptide. A conservative amino acid substitution may encompass non-naturally occurring amino acid residues, which are typically incorporated by chemical peptide synthesis rather than by synthesis in biological systems. These include, but are not limited to, peptidomimetics and other reversed or inverted forms of amino acid moieties.

Naturally occurring residues may be divided into classes based on common side chain properties: 1) hydrophobic: norleucine, Met, Ala, Val, Leu, Ile; 2) neutral hydrophilic: Cys, Ser, Thr, Asn, Gln; 3) acidic: Asp, Glu; 4) basic: His, Lys, Arg; 5) residues that influence chain orientation: Gly, Pro; and 6) aromatic: Trp, Tyr, Phe.

For example, non-conservative substitutions may involve the exchange of a member of one of these classes for a member from another class. Such substituted residues may be introduced into regions of a human antibody that are homologous with non-human antibodies, or into the non-homologous regions of the molecule.

In making substitutions, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein, in certain instances, is understood in the art. Kyte et al., J. Mol. Biol., 157:105-131 (1982). It is known that in certain instances, certain amino acids may be substituted for other amino acids having a similar hydropathic index or score and still retain a similar biological activity. In making changes based upon the hydropathic index, in certain embodiments, the substitution of amino acids whose hydropathic indices are within ±0.2 is included. In certain embodiments, those which are within ±0.1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0 ±0.1); glutamate (+3. 0±0.1); serine (+0.3); asparagine (+0.2); glutamine (+0.2); glycine (0); threonine (−0.4); proline (−0.5 ±0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±0.2 is included, in certain embodiments, those which are within ±1 are included, and in certain embodiments, those within ±0.5 are included. One may also identify epitopes from primary amino acid sequences on the basis of hydrophilicity.

A skilled artisan will be able to determine suitable variants of a polypeptide as set forth herein using well-known techniques. In certain embodiments, one skilled in the art may identify suitable areas of the molecule that may be changed without destroying activity by targeting regions not believed to be important for activity. In certain embodiments, one can identify residues and portions of the molecules that are conserved among similar polypeptides. In certain embodiments, even areas that may be important for biological activity or for structure may be subject to conservative amino acid substitutions without destroying the biological activity or without adversely affecting the polypeptide structure.

Additionally, in certain embodiments, one skilled in the art can review structure-function studies identifying residues in similar polypeptides that are important for activity or structure. In view of such a comparison, in certain embodiments, one can predict the importance of amino acid residues in a protein that correspond to amino acid residues which are important for activity or structure in similar proteins. In certain embodiments, one skilled in the art may opt for chemically similar amino acid substitutions for such predicted important amino acid residues.

In certain embodiments, one skilled in the art can also analyze the three-dimensional structure and amino acid sequence in relation to that structure in similar polypeptides. In certain embodiments, in view of such information, one skilled in the art may predict the alignment of amino acid residues of an antibody with respect to its three dimensional structure. In certain embodiments, one skilled in the art may choose not to make radical changes to amino acid residues predicted to be on the surface of the protein, since such residues may be involved in important interactions with other molecules. Moreover, in certain embodiments, one skilled in the art may generate test variants containing a single amino acid substitution at each desired amino acid residue. In certain embodiments, the variants can then be screened using activity assays known to those skilled in the art. In certain embodiments, such variants could be used to gather information about suitable variants. For example, in certain embodiments, if one discovered that a change to a particular amino acid residue resulted in destroyed, undesirably reduced, or unsuitable activity, variants with such a change may be avoided. In other words, in certain embodiments, based on information gathered from such routine experiments, one skilled in the art can readily determine the amino acids where further substitutions should be avoided either alone or in combination with other mutations.

A number of scientific publications have been devoted to the prediction of secondary structure. See, e.g., Moult J., Curr. Op. in Biotech., 7(4):422-427 (1996), Chou et al., Biochemistry, 13(2):222-245 (1974); Chou et al., Biochemistry, 113(2):211-222 (1974); Chou et al., Adv. Enzymol. Relat. Areas Mol. Biol., 47:45-148 (1978); Chou et al., Ann. Rev. Biochem., 47:251-276 and Chou et al., Biophys. J., 26:367-384 (1979). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The growth of the protein structural database (PDB) has provided enhanced predictability of secondary structure, including the potential number of folds within a polypeptide's structure. See, e.g., Holm et al., Nucl. Acid. Res., 27(1):244-247 (1999). It has been suggested (Brenner et al., Curr. Op. Struct. Biol., 7(3):369-376 (1997)) that there are a limited number of folds in a given polypeptide or protein and that once a critical number of structures have been resolved, structural prediction will become dramatically more accurate.

Additional methods of predicting secondary structure include “threading” (see, e.g., Jones, D., Curr. Opin. Struct. Biol., 7(3):377-87 (1997); Sippl et al., Structure, 4(1):15-19 (1996)), “profile analysis” (see, e.g., Bowie et al., Science, 253:164-170 (1991); Gribskov et al., Meth. Enzym., 183:146-159 (1990); Gribskov et al., Proc. Nat. Acad. Sci., 84(13):4355-4358 (1987)), and “evolutionary linkage” (see, e.g., Holm et al., Nucl. Acid. Res., 27(1):244-247 (1999), and Brenner et al., Curr. Op. Struct. Biol., 7(3):369-376 (1997)).

“Percent identity” or “% identity,” with reference to nucleic acid sequences, refers to the percentage of identical nucleotides between at least two polynucleotide sequences aligned using the Basic Local Alignment Search Tool (BLAST) engine. See Tatusova et al. (1999) FEMS Microbiol Lett. 174:247-250. The BLAST engine (version 2.2.10) is provided to the public by the National Center for Biotechnology Information (NCBI), Bethesda, Md. To align two polynucleotide sequences, the “Blast 2 Sequences” tool can be used.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, the terms “treat,” and “prevent” as well as words stemming therefrom, do not necessarily imply 100% or complete treatment or prevention. Rather, there are varying degrees of treatment or prevention of which one of ordinary skill hi the art recognizes as having a potential benefit or therapeutic effect. In this respect, the methods of the present invention can provide any amount of any level of treatment or prevention of a disease or medical condition in a mammal. Furthermore, the treatment or prevention provided by the method can include treatment or prevention of one or more conditions or symptoms of the disease or medical condition. For example, with regard to methods of treating obesity, the method in some embodiments, achieves a decrease in food intake by or fat levels in a subject. Also, for purposes herein, “prevention” can encompass delaying the onset of the disease, or a symptom or condition thereof. The term “treating” includes prophylaxis of the specific disorder or condition, or alleviation of the symptoms associated with a specific disorder or condition and/or preventing or eliminating said symptoms. For example, as used herein the term “treating diabetes” refers in general to altering glucose blood levels in the direction of normal levels and may include increasing or decreasing blood glucose levels depending on a given situation.

As used herein an “effective” amount or a “therapeutically effective amount” of the isolated Angptl-FLD polypeptide of the invention refers to a nontoxic but sufficient amount of the peptide to provide the desired effect. For example one desired effect would be the prevention or treatment of hypoglycemia, as measured, for example, by an increase in blood glucose level. An alternative desired effect for the isolated Angptl4-FLD polypeptide of the invention would include treating hyperglycemia, e.g., as measured by a change in blood glucose level closer to normal, or inducing weight loss/preventing weight gain, e.g., as measured by reduction in body weight, or preventing or reducing an increase in body weight, or normalizing body fat distribution. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term, “parenteral” means not through the alimentary canal but by some other route, e.g., subcutaneous, intramuscular, intraspinal, or intravenous.

The term “isolated” as used herein means having been removed from its natural environment. In some embodiments, the analog is made through recombinant methods and the analog is isolated from the host cell. The term “isolated,” relates to the isolation of a molecule or compound in a form that is substantially free of contaminants normally associated with the molecule or compound in a native or natural environment and means having been increased in purity as a result of being separated from other components of the original composition. The term “isolated polypeptide” is used herein to describe a polypeptide which has been separated from other compounds including, but not limited to nucleic acid molecules, lipids and carbohydrates. An “isolated polypeptide may be found in a pharmaceutical formulation also including a pharmaceutically acceptable diluent.

The term “agent” refers to a chemical compound, a mixture of chemical compounds, a biological macromolecule, or an extract made from biological materials.

A “therapeutic agent” refers to an agent that may be administered in vivo to bring about a therapeutic and/or prophylactic/preventative effect.

A “therapeutic polypeptide” refers to polypeptide that may be administered in vivo to bring about a therapeutic and/or prophylactic/preventative effect.

The terms “isolated nucleic acid” and “isolated polynucleotide” are used interchangeably and refer to a polynucleotide of genomic, cDNA, or synthetic origin or some combination thereof. An “isolated polynucleotide” (1) is not associated with all or a portion of a polynucleotide in which the “isolated polynucleotide” is found in nature, (2) is linked to a polynucleotide to which it is not linked in nature, or (3) does not occur in nature as part of a larger sequence.

The term “diabetes mellitus” or “diabetes” refers to a disease or condition that is generally characterized by metabolic defects in production and utilization of glucose which result in the failure to maintain appropriate blood sugar levels in the body. Diabetes may be classified as type 1 diabetes (generally due to the absence of insulin production due to autoimmune destruction of pancreatic (3-cells) or type 2 diabetes (T2D; generally due to existing insulin levels in the body that are inadequate to normalize plasma glucose levels, and believed to primarily result from a condition known as “insulin resistance,” in which there is a decreased biological response to normal concentrations of circulating insulin). In some cases, diabetes may also be caused by any number of other conditions, including pregnancy.

A “pre-diabetic individual,” when used to compare with a sample from a subject, refers to an adult with a fasting blood glucose level greater than about 110 mg/dl but less than about 126 mg/dl or a 2 hour post-load glucose (PG) reading of greater than about 140 mg/dl but less thanabout 200 mg/dl. A “diabetic individual,” when used to compare with a sample from a subject, refers to an adult with a fasting blood glucose level greater than about 126 mg/dl or a 2 hour PG reading of greater than about 200 mg/dl.

### The Embodiments

Isolated Angptl4-FLD polypeptides and Variants

In various embodiments, the present invention provides compositions comprising the C-terminal fibrinogen-like domain (FLD) of Angptl4, and variants thereof, which have been discovered to elevate intracellular cAMP levels and induce lipolysis in adipocytes.

In various embodiments, the present invention provides novel compositions and methods for preventing and reducing obesity, and improving insulin sensitivity of obese and type 2 diabetes patients.

In various embodiments, the invention provides compositions and methods for increasing Angptl4-FLD, or a variant thereof, in circulation in a subject, thereby reducing adiposity without elevating plasma TG levels.

In an exemplary embodiment, the invention provides compositions and methods for increasing Angptl4-FLD, or a variant thereof, in circulation in a subject, thereby improving glucose homeostasis.

At least in part, the present invention is based on the discovery that Angptl4, and variants thereof comprising at least a subsequence of the FLD domain of Angptl4, curtail the development of obesity, insulin resistance, diabetes, hypertriglyceridemia, and hepatic steatosis, and increases energy expenditure.

Thus, in an exemplary embodiment, the invention provides isolated polypeptides comprising a variant of the naturally occuring sequence of Angptl4, which includes at least a subsequence of the FLD domain of Angptl4. An exemplary naturally occuring Angplt sequence is:

SEQ. ID. NO.: 1
MSGAPTAGAA LMLCAATAVL LSAQGGPVQS KSPRFASWDE
MNVLAHGLLQ LGQGLREHAE RTRSQLSALE RRLSACGSAC
QGTEGSTDLP LAPESRVDPE VLHSLQTQLK AQNSRIQQLF
HKVAQQQRHL EKQHLRIQHL QSQFGLLDHK HLDHEVAKPA
RRKRLPEMAQ PVDPAHNVSR LHRLPRDCQE LFQVGERQSG
LFEIQPQGSP PFLVNCKMTS DGGWTVIQRR HDGSVDFNRP
WEAYKAGFGD PHGEFWLGLE KVHSITGDRN SRLAVQLRDW
DGNAELLQFS VHLGGEDTAY SLQLTAPVAG QLGATTVPPS
GLSVPFSTWD QDHDLRRDKN CAKSLSGGWW FGTCSHSNLN
GQYFRSIPQQ RQKLKKGIFW KTWRGRYYPL QATTMLIQPM AAEAAS.

In some embodiments, the invention provides an isolated variant of SEQ. ID. NO.: 1 in which at least amino acids 38-165 are deleted.

In various embodiments, the polypeptide of the invention does not include a signal peptide sequence.

In various embodiments, the polypeptide of the invention includes amino acids 165-406 of SEQ. ID. NO.: 1. In various embodiments, the invention provides an isolated polypeptide having a sequence which has at least about 90%, at least about 91%, at least about 92%, at least about 93%, at least about 94%, at least about 95%, at least about 96%, at least about 97%, at least about 98% or at least about 99% sequence identity with a polypeptide including amino acids 165-406 of SEQ. ID. NO.: 1. In various embodiments, the invention provides a polypeptide in which the sequence of amino acids 165-406 of SEQ. ID. NO.: 1 is the only sequence in the polypeptide corresponding to SEQ. ID. NO.: 1.

In various embodiments, the polypeptide of the invention comprises amino acids 184-406 of SEQ. ID. NO.: 1. In various embodiments, the invention provides an isolated polypeptide having a sequence which has at least about 90%, at least about 91%, at least about 92%, at least about 93%, at least about 94%, at least about 95%, at least about 96%, at least about 97%, at least about 98% or at least about 99% sequence identity with a polypeptide including amino acids 184-406 of SEQ. ID. NO.: 1. In various embodiments, the invention provides a polypeptide in which the sequence of amino acids 184-406 of SEQ. ID. NO.: 1 is the only sequence in the polypeptide corresponding to SEQ. ID. NO.: 1.

In various embodiments, the invention provides an isolated polypeptide having a sequence which has at least about 90%, at least about 91%, at least about 92%, at least about 93%, at least about 94%, at least about 95%, at least about 96%, at least about 97%, at least about 98% or at least about 99% sequence identity with a polypeptide of SEQ. ID. NO.: 2:

SEQ. ID. NO.: 2
MSGAPTAGAA LMLCAATAVL LSAQGGPVQS KSPRFASW(Xaa)ₙ
PEMAQ PVDPAHNVSR LHRLPRDCQE LFQVGERQSG LFEIQPQGSP
PFLVNCKMTS DGGWTVIQRR HDGSVDFNRP WEAYKAGFGD
PHGEFWLGLE KVHSITGDRN SRLAVQLRDW DGNAELLQFS
VHLGGEDTAY SLQLTAPVAG QLGATTVPPS GLSVPFSTWD
QDHDLRRDKN CAKSLSGGWW FGTCSHSNLN GQYFRSIPQQ
RQKLKKGIFW KTWRGRYYPL QATTMLIQPM AAEAAS.

In SEQ. ID. NO.: 2, Xaa is a naturally occurring amino acid or is an uncoded amino acid. The index n includes the integers 0, 1, 2, 3, 4, 5, 6, 7, 8, 9, 10 or higher. In an exemplary embodiment, (Xaa)ₙ is a linker. In an exemplary embodiment, (Xaa)ₙ is an amino acid that provides a locus for conjugation of a heterologous moiety to the reamainder of the polypeptide according to SEQ. ID. NO.: 2, or (Xaa)ₙ represents that amino acid and heterologous moiety conjugate.

In an exemplary embodiment, the isolated polypeptide of the invention is functionalized at the N-terminus with a bond or a linker to a heterologous moiety. In an exemplary embodiment, the isolated polypeptide of the invention is functionalized at the C-terminus with a bond or a linker to a heterologous moiety. In an exemplary embodiment, the isolated polypeptide of the invention is functionalized at the N-terminus and C-terminus with a bond or a linker to a heterologous moiety. When the isolated polypeptides of the invention include more than one heterologous moiety, each heterologous moiety is independently selected.

In an exemplary embodiment, the polypeptide of the invention includes one or more C- and or N-terminal “tags” to facilitate isolation or purification of the polypeptide. Such tags and methods of placing and using them are recognized in the art. Exemplary tags include, without limitation, FLAG-tag, histidine tag and h15uman influenza hemagglutinin (HA)-tag.

Modifications that Improve DPP-IV Resistance

In various embodiments, the isolated Angptl4-FLD polypeptide comprises one or two modifications which increase the peptide's resistance to dipeptidyl peptidase IV (DPP IV) cleavage. In exemplary embodiments, the amino acid at position 2 of the isolated polypeptide is substituted with one of: D-serine, D-alanine, valine, glycine, N-methyl serine, N-methyl alanine, or amino isobutyric acid (AIB). In exemplary embodiments, the amino acid at position 1 of the isolated polypeptide is substituted with one of: D-histidine, desaminohistidine, hydroxyl-histidine, acetyl-histidine, homo-histidine, N-methyl histidine, alpha-methyl histidine, imidazole acetic acid, or alpha, alpha-dimethyl imidiazole acetic acid (DMIA). In some aspects, the isolated polypeptide comprising an amino acid modification which increases resistance to DPP IV further comprises an intramolecular bridge or alpha, alpha di-substituted amino acid, and optionally an amino acid modification which selectively reduces the activity at the glucagon receptor.

Modifications that Reduce Degradation

In exemplary embodiments, any of the isolated polypeptides of the present invention can be further modified to improve stability of the peptide by modifying one or more amino acids of SEQ ID NO: 2 to reduce degradation of the polypeptide over time, especially in acidic or alkaline buffers.

In some embodiments, the therapeutic polypeptide comprises a modification which prevents oxidative degradation of the peptide. For example, a methionine residue is modified, e.g. by deletion or substitution. In some embodiments, a Met is substituted with leucine, isoleucine or norleucine.

In some embodiments, the isolated polypeptide comprises one or more modifications that reduce degradation through deamidation of Gln. In some aspects, a Gln of the isolated polypeptide is modified, e.g. by deletion or substitution. In some embodiments, a Gln is substituted with Ser, Thr, Ala or AIB. In some embodiments a Gln is substituted with Lys, Arg, Orn, or Citrulline.

In some embodiments, the isolated polypeptide comprises an amino acid modification which reduces degradation through dehydration of Asp to form a cyclic succinimide intermediate followed by isomerization to iso-aspartate. Accordingly, in some aspects, an Asp of the isolated polypeptide is modified, e.g. by deletion or substitution. In some embodiments, an Asp of the isolated polypeptide is substituted with Glu, homoglutamic acid or homocysteic acid.

Modifications that Enhance Solubility

In another embodiment, the solubility of any of the isolated polypeptides is improved by one or more amino acid substitutions and/or additions that introduce a charged amino acid into the C-terminal portion of the peptide. Optionally, one, two or three charged amino acids are introduced within the C-terminal portion. In some embodiments of the present invention, one or more native amino acid(s) are substituted with one or two charged amino acids, and/or in further embodiments one to three charged amino acids are also added to the C-terminus of the isolated polypeptide. In exemplary embodiments, one, two or all of the charged amino acids are negative-charged or acidic amino acids. In some embodiments, the negative-charged or acidic amino acids are aspartic acid or glutamic acid. In other embodiments, one, two, three or all of the charged amino acids are positively charged. Such modifications increase solubility, e.g. provide at least 2-fold, 5-fold, 10-fold, 15-fold, 25-fold, 30-fold or greater solubility relative to native Angptl4-FLD at a given pH between about 5.5 and 8, e.g., pH 7, when measured after 24 hours at 25 ° C.

The addition of a hydrophilic moiety also can enhance solubility of the isolated polypeptide. Hydrophilic moieties and conjugation thereof to peptides is further described herein. See, “Conjugates.” In exemplary embodiments, the therapeutic polypeptide is conjugated to a hydrophilic moiety, e.g., polyethylene glycol, at position 16, 17, 20, 21, 24 or 29 of the therapeutic polypeptide, within a C-terminal extension, and/or at the C-terminal amino acid of the peptide. Such modifications also enhance the duration of action or half-life of the polypeptide in circulation.

### Other Modifications

Additional modifications, e.g., conservative substitutions, may be made to the isolated polypeptide. In various embodiments, the modification does not substantially decrese the activity of the modified polypeptide relative to the unmodified polypeptide.

### Conjugates

In an exemplary embodiment, the present invention further provides conjugates of the isolated Angptl4-FLD polypeptide. In some aspects, the conjugate comprises an isolated Angptl4-FLD conjugated to a second peptide, e.g., a glucagon antagonist peptide. In some aspects, the invention provides a conjugate comprising the isolated Angptl-FLD polypeptide of the invention conjugated to a heterologous moiety. In some aspects, the conjugate comprises isolated Angptl4-FLD conjugated to a glucagon antagonist peptide and at least one of the peptides is conjugated to a heterologous moiety.

The conjugation between the two peptides or between the peptide and heterologous moiety may involve covalent bonds, non-covalent bonds, or both types of bonds. In some aspects, the covalent bonds are any of the covalent linkages described herein (e.g., disulfide bonds, lactam bridges, olefin metathesis, and the like). In some aspects, the covalent bonds are peptide bonds. In specific embodiments in which the conjugation involves peptide bonds, the conjugate may be a fusion peptide comprising the isolated Angptl4-FLD polypeptide and a second peptide (e.g.,a glucagon antagonist peptide). In various embodiments, the fusion peptide optionally a heterologous moiety, e.g., a Fc receptor, or portion thereof.

In various embodiments, the isolated Angtl4-FLD polypeptide is conjugated to the second peptide through non-covalent linkages, e.g., electrostatic interactions, hydrogen bonds, van der Waals interactions, salt bridges, hydrophobic interactions, and the like.

The conjugation of the isolated Angbtl4-FLD polypeptide to a second peptide and/or to the heterologous moiety may be indirect or direct conjugation, the former of which may involve a linker or spacer. Suitable linkers and spacers are known in the art and include, but not limited to, any of the linkers or spacers described herein under the section “Linkages.”

### Heterologous Moieties

As used herein, the term “heterologous moiety” is synonymous with the term “conjugate moiety” and refers to any molecule (chemical or biochemical, naturally-occurring or non-coded) which is different from the isolated Angptl-FLD polypeptide of the invention (or a second peptide) to which it is attached. Exemplary conjugate moieties that can be linked to any of the analogs described herein include but are not limited to a heterologous peptide or polypeptide (including for example, a plasma protein), a targeting agent, an immunoglobulin or portion thereof (e.g., variable region, CDR, or Fc region), a diagnostic label such as a radioisotope, fluorophore or enzymatic label, a polymer including water soluble polymers, or other therapeutic or diagnostic agents. In some embodiments a conjugate is provided comprising a peptide of the peptide combination and a plasma protein, wherein the plasma protein is selected from the group consisting of albumin, transferin, fibrinogen and globulins. In some embodiments the plasma protein moiety of the conjugate is albumin or transferin. The conjugate in some embodiments comprises one or more of the peptides of the peptide combinations described herein and one or more of: a peptide (which is distinct from the isolated Angptl-FLD polypeptide of the invention described herein), a polypeptide, a nucleic acid molecule, an antibody or fragment thereof, a polymer, a quantum dot, a small molecule, a toxin, a diagnostic agent, a carbohydrate, an amino acid.

With respect to conjugates between isolated Angptl4-FLD polypeptide and a polymer, the polymer may be one that is naturally occurring. In various embodiments, the polymer naturally includes a carbonyl moiety or may be one which was derivatized to comprise the carbonyl bearing the carbonyl. In these embodiments, the polymer is attached to the isolated Angptl4-FLD polypeptide through an amide bond.

The polymer, without limitation, may be any of: polyamides, polycarbonates, polyalkylenes and derivatives thereof including, polyalkylene glycols, polyalkylene oxides, polyalkylene terepthalates, polymers of acrylic and methacrylic esters, including poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), polyvinyl polymers including polyvinyl alcohols, polyvinyl ethers, polyvinyl esters, polyvinyl halides, poly(vinyl acetate), and polyvinylpyrrolidone, polyglycolides, polysiloxanes, polyurethanes and co-polymers thereof, celluloses including alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt, polypropylene, polyethylenes including poly(ethylene glycol), poly(ethylene oxide), and poly(ethylene terephthalate), and polystyrene.

The polymer can be a biodegradable polymer, including a synthetic biodegradable polymer (e.g., polymers of lactic acid and glycolic acid, polyanhydrides, poly(ortho)esters, polyurethanes, poly(butic acid), poly(valeric acid), and poly(lactide-cocaprolactone)), and a natural biodegradable polymer (e.g., alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins (e.g., zein and other prolamines and hydrophobic proteins)), as well as any copolymer or mixture thereof. In general, these materials degrade either by enzymatic hydrolysis or exposure to water in vivo, by surface or bulk erosion.

The polymer can be a bioadhesive polymer, such as a bioerodible hydrogel described by H. S. Sawhney, C. P. Pathak and J. A. Hubbell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

In some aspects, the polymer is a water-soluble polymer. Suitable water-soluble polymers are known in the art and include, for example, polyvinylpyrrolidone, hydroxypropyl cellulose (HPC; Klucel), hydroxypropyl methylcellulose (HPMC; Methocel), nitrocellulose, hydroxypropyl ethylcellulose, hydroxypropyl butylcellulose, hydroxypropyl pentylcellulose, methyl cellulose, ethylcellulose (Ethocel), hydroxyethyl cellulose, various alkyl celluloses and hydroxyalkyl celluloses, various cellulose ethers, cellulose acetate, carboxymethyl cellulose, sodium carboxymethyl cellulose, calcium carboxymethyl cellulose, vinyl acetate/crotonic acid copolymers, poly-hydroxyalkyl methacrylate, hydroxymethyl methacrylate, methacrylic acid copolymers, polymethacrylic acid, polymethylmethacrylate, maleic anhydride/methyl vinyl ether copolymers, poly vinyl alcohol, sodium and calcium polyacrylic acid, polyacrylic acid, acidic carboxy polymers, carboxypolymethylene, carboxyvinyl polymers, polyoxyethylene polyoxypropylene copolymer, polymethylvinylether co-maleic anhydride, carboxymethylamide, potassium methacrylate divinylbenzene co-polymer, polyoxyethyleneglycols, polyethylene oxide, and derivatives, salts, and combinations thereof.

In specific embodiments, the polymer is a polyalkylene glycol, including, for example, polyethylene glycol (PEG).

In some embodiments, the polymer has a molecular weight of about 100 kDa or less, e.g., about 90 kDa or less, about 80 kDa or less, about 70 kDa or less, about 60 kDa or less, about 50 kDa or less, about 40 kDa or less. Accordingly, the polymer can have a molecular weight of about 35 kDa or less, about 30 kDa or less, about 25 kDa or less, about 20 kDa or less, about 15 kDa or less, about 10 kDa or less, about 5 kDa or less, or about 1 kDa.

### Multimers

In some embodiments in which the isolated Angptl4-FLD polypeptide is conjugated to a second isolated Angptl4-FLD polypeptide, and the conjugate is not a fusion peptide, the conjugate is a multimer or dimer comprising the peptides of the peptide combinations. The conjugate may be a hetero-multimer or heterodimer comprising the isolated Angptl4-FLD polypeptide conjugated to the second isolated Angptl4-FLD polypeptide. In certain embodiments, the linker connecting the two (or more) peptides is PEG, e.g., a 5 kDa PEG, 20 kDa PEG. In some embodiments, the linker is a disulfide bond. For example, each monomer of the dimer may comprise a Cys residue (e.g., a terminal or internally positioned Cys) and the sulfur atom of each Cys residue participates in the formation of the disulfide bond. In some aspects, the monomers are connected via terminal amino acids (e.g., N-terminal or C-terminal), via internal amino acids, or via a terminal amino acid of at least one monomer and an internal amino acid of at least one other monomer. In specific aspects, the monomers are not connected via an N-terminal amino acid. In some aspects, the monomers of the multimer are attached together in a “tail-to-tail” orientation in which the C-terminal amino acids of each monomer are attached together.

### Linkages /Linkers

The following two sections on linkages provide description for linking a peptide to a heterologous moiety or for dimer or multimer formation. The skilled artisan will recognize that the teachings of one type of conjugate may be applicable to the other type.

### Linkages—Peptide to Heterologous Moieties

The formation of a conjugate of the invention, in some embodiments, involves direct covalent linkage by reacting targeted amino acid residues of the isolated Angptl4-FLD polypeptide with an organic derivatizing agent that is capable of reacting with selected side chains or the N- or C-terminal residues of these targeted amino acids. Reactive groups on the analog or conjugate moiety include, e.g., an aldehyde, amino, ester, thiol, alpha-haloacetyl, maleimido or hydrazino group. Derivatizing agents include, for example, maleimidobenzoyl sulfosuccinimide ester (conjugation through cysteine residues), N-hydroxysuccinimide (through lysine residues), glutaraldehyde, succinic anhydride or other agents known in the art. Alternatively, the conjugate moieties can be linked to the analog indirectly through intermediate carriers, such as polysaccharide or polypeptide carriers. Examples of polysaccharide carriers include aminodextran. Examples of suitable polypeptide carriers include polylysine, polyglutamic acid, polyaspartic acid, co-polymers thereof, and mixed polymers of these amino acids and others, e.g., serines, to confer desirable solubility properties on the resultant loaded carrier.

Cysteinyl residues are most commonly reacted with alpha-haloacetates (and corresponding amines), such as chloroacetic acid, chloroacetamide to give carboxymethyl or carboxyamidomethyl derivatives. Cysteinyl residues also are derivatized by reaction with bromotrifluoroacetone, alpha-bromo-beta-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino-terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

In some embodiments, arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

Exemplary specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively.

In various embodmiments, carboxyl side groups (aspartyl or glutamyl) are selectively modified by reaction with carbodiimides, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Other exemplary modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)), deamidation of asparagine or glutamine, acetylation of the N-terminal amine, and/or amidation or esterification of the C-terminal carboxylic acid group.

An exemplary type of covalent modification involves chemically or enzymatically coupling glycosides to the analog. Sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of tyrosine, or tryptophan, or (f) the amide group of glutamine. Exemplary methods are described in WO87/05330 published 11 Sep. 1987, and in Aplin and Wriston, CRC Crit. Rev. Biochem., pp. 259-306 (1981).

In some embodiments, the peptide is conjugated to a heterologous moiety via covalent linkage between a side chain of an amino acid of the isolated Angptl4-FLD polypeptide and the heterologous moiety. In some embodiments, the isolated Angptl4-FLD polypeptide is conjugated to a heterologous moiety via the side chain of an amino acid, a position within a C-terminal extension, or the C-terminal amino acid, or a combination of these positions. In some aspects, the amino acid covalently linked to a heterologous moiety (e.g., the amino acid comprising a heterologous moiety) is a Cys, Lys, Orn, homo-Cys, or Ac-Phe, and the side chain of the amino acid is covalently bonded to a heterologous moiety.

In some embodiments, the conjugate comprises a linker that joins the isolated Angptl4-FLD polypeptideto the heterologous moiety. In some aspects, the linker comprises a chain of atoms from 1 to about 60, or 1 to 30 atoms or longer, 2 to 5 atoms, 2 to 10 atoms, 5 to 10 atoms, or to 20 atoms long. In some embodiments, the chain atoms are all carbon atoms. In some embodiments, the chain atoms in the backbone of the linker are selected from the group consisting of C, O, N, and S. Chain atoms and linkers may be selected according to their expected solubility (hydrophilicity) so as to provide a more soluble conjugate. In some embodiments, the linker provides a functional group that is subject to cleavage by an enzyme or other catalyst or hydrolytic conditions found in the target tissue or organ or cell. In some embodiments, the length of the linker is long enough to reduce the potential for steric hindrance. If the linker is a covalent bond or a peptidyl bond and the conjugate is a polypeptide, the entire conjugate can be a fusion protein. Such peptidyl linkers may be any length. Exemplary linkers are from about 1 to 50 amino acids in length, 5 to 50, 3 to 5, 5 to 10, 5 to 15, or 10 to 30 amino acids in length. Such fusion proteins may alternatively be produced by recombinant genetic engineering methods known to one of ordinary skill in the art.

In some embodiments, the heterologous moiety is attached via non-covalent or covalent bonding to the isolated Angptl4-FLD polypeptide. In certain aspects, the heterologous moiety is attached to the isolated Angptl4-FLD polypeptide via a linker. Linkage can be accomplished by covalent chemical bonds, physical forces such electrostatic, hydrogen, ionic, van der Waals, or hydrophobic or hydrophilic interactions. A variety of non-covalent coupling systems may be used, including biotin-avidin, ligand/receptor, enzyme/substrate, nucleic acid/nucleic acid binding protein, lipid/lipid binding protein, cellular adhesion molecule partners; or any binding partners or fragments thereof which have affinity for each other.

### Linkages—Dimers and Multimers

In some embodiments, the components of the conjugate are linked together using standard linking agents and procedures known to those skilled in the art. For example, in some aspects, the components of the conjugate are fused through one or more peptide bonds, with or without a spacer, e.g., a peptide or amino acid spacer. In various embodiments, the components of the conjugate are linked together through chemical conjugation. In some embodiments, the components of the conjugate are chemically conjugated together through a linking moiety. In an exemplary embodiment, the linking moiety is directly conjugated to each component, or, in alternative aspects, are indirectly conjugated to each peptide through a spacer.

In some aspects, the isolated Angptl4-FLD polypeptide and a second isolated polypeptide are linked together in a “tail-to-tail” orientation in which the C-terminal amino acids of the peptides are conjugated together. In some aspects, the therapeutic polypeptide and second peptide of the conjugate are linked together via the side chains of internal amino acids on each peptide. In some aspects, the isolated Angptl4-FLD polypeptide and second peptide of the conjugate are linked together via a C-terminal amino acid of one peptide and an internal amino acid of another peptide.

In some embodiments, two peptides of the conjugate are directly linked together and do not comprise a linking moiety. In some embodiments, the two peptides of the conjugate are linked together by conjugating both of the peptides to a single linking moiety that comprises at least two reactive groups, e.g., a bifunctional linker, a bifunctional spacer. In some embodiments, the two peptides of the conjugate are linked together by indirectly conjugating one or both of the peptides to the single linking moiety through a spacer.

In some embodiments, the C-terminal of the isolated Angptl4-FLD polypeptide of the conjugate is modified to comprise a natural or nonnatural amino acid with a nucleophilic side chain. In exemplary embodiments, the C-terminal amino acid of the isolated Angptl-FLD polypeptide of the invention is selected from the group consisting of lysine, ornithine, serine, cysteine, and homocysteine. In some embodiments, the C-terminal amino acid of one or more peptides of the conjugate is modified to comprise a natural or nonnatural amino acid with an electrophilic side chain such as, for example, Asp and Glu. In some embodiments, the C-terminal amino acids of both peptides of the conjugate comprise side chains that are nulceophilic. In some embodiments, the C-terminal amino acids of both peptides comprise side chains that are electrophilic. In some embodiments, the C-terminal amino acid of one peptide of the conjugate comprises a side chain that is nulceophilic, and the C-terminal amino acid of the other peptide of the conjugate comprises a side chain that is electrophilic.

In some embodiments, the two peptides of the conjugate are linked together by directly conjugating the C-terminal amino acids of the peptides to one another with a linking moiety. In some embodiments, the two peptides of the conjugate are linked by directly conjugating the C-terminal amino acid side chain of one peptide to the C-terminal amino acid side chain of the other peptide. In some of these embodiments, the C-terminal amino acid of one peptide comprises a nucleophilic side chain and the C-terminal amino acid of the other peptide comprises an electrophilic side chain. In some of these embodiments, both C-terminal amino acids comprise thiol side chains and linkage occurs through a disulfide bond.

In some embodiments, two peptides of the conjugate are linked through a nucleophilic side chain of the C-terminal amino acid of one peptide to the alpha-carboxyl group of the C-terminal amino acid on the other peptide.

In some embodiments, the two peptides of the conjugate are linked together by conjugating the C-terminal amino acid side chains of both of the peptides to a linking moiety that comprises at least two reactive groups before conjugation to the peptides. In some embodiments, the linking moiety is a bifunctional linker and comprises only two reactive groups before conjugation to the peptides. In embodiments where the two peptides of the composition both have C-terminal amino acids with electrophilic side chains, the linking moiety comprises two of the same or two different nucleophilic groups (e.g., amine, hydroxyl, thiol) before conjugation to the peptides. In embodiments where the two peptides of the composition both have C-terminal amino acids with nucleophilic side chains, the linking moiety comprises two of the same or two different electrophilic groups (e.g. carboxyl group, activated form of a carboxyl group, compound with a leaving group) before conjugation to the peptides. In embodiments where one peptide of the composition has a C-terminal amino acid with a nucleophilic side chain and the other peptide of the composition has a C-terminal amino acid with an electrophilic side chain, the linking moiety comprises one nucleophilic group and one electrophilic group before conjugation to the peptides. In some embodiments where one or more of the two peptides of the composition are conjugated to each other through their C-terminal alpha-carboxyl groups, the linking moiety comprising two nucleophilic groups before conjugation to the peptides.

Pharmaceutical Salts

In some embodiments, the isolated Angptl4-FLD polypeptide of the present invention is present in the composition (or conjugate) in the form of a salt, e.g., a pharmaceutically acceptable salt. As used herein the term “pharmaceutically acceptable salt” refers to salts of compounds that retain the biological activity of the parent compound, and which are not biologically or otherwise undesirable. Such salts can be prepared in situ during the final isolation and purification of the analog, or separately prepared by reacting a free base function with a suitable acid. Many of the compounds disclosed herein are capable of forming acid and/or base salts by virtue of the presence of amino and/or carboxyl groups or groups similar thereto.

Pharmaceutically acceptable acid addition salts may be prepared from inorganic and organic acids. Representative acid addition salts include, but are not limited to acetate, adipate, alginate, citrate, aspartate, benzoate, benzenesulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate, and undecanoate. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Examples of acids which can be employed to form pharmaceutically acceptable acid addition salts include, for example, an inorganic acid, e.g., hydrochloric acid, hydrobromic acid, sulphuric acid, and phosphoric acid, and an organic acid, e.g., oxalic acid, maleic acid, succinic acid, and citric acid.

Basic addition salts also can be prepared in situ during the final isolation and purification of the source of salicylic acid, or by reacting a carboxylic acid-containing moiety with a suitable base such as the hydroxide, carbonate, or bicarbonate of a pharmaceutically acceptable metal cation or with ammonia or an organic primary, secondary, or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylammonium, dimethylammonium, trimethylammonium, triethylammonium, diethylammonium, and ethylammonium, amongst others. Other representative organic amines useful for the formation of base addition salts include, for example, ethylenediamine, ethanolamine, diethanolamine, piperidine, piperazine, and the like. Salts derived from organic bases include, but are not limited to, salts of primary, secondary and tertiary amines.

Further, basic nitrogen-containing groups in the isolated Angptl4-FLD polypeptide can be quaternized using lower alkyl halides such as methyl, ethyl, propyl, and butyl chlorides, bromides, and iodides; long chain halides such as decyl, lauryl, myristyl, and stearyl chlorides, bromides, and iodides; arylalkyl halides like benzyl and phenethyl bromides and others. Water or oil-soluble or dispersible products are thereby obtained.

### Pharmaceutical Formulations

In accordance with some embodiments, the isolated Angptl4-FLD polypeptide of the present invention is a pharmaceutical composition and further comprises a pharmaceutically acceptable carrier. The pharmaceutical composition can comprise any pharmaceutically acceptable ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents.

In some embodiments, the pharmaceutical composition comprises any one or a combination of the following components: acacia, acesulfame potassium, acetyltributyl citrate, acetyltriethyl citrate, agar, albumin, alcohol, dehydrated alcohol, denatured alcohol, dilute alcohol, aleuritic acid, alginic acid, aliphatic polyesters, alumina, aluminum hydroxide, aluminum stearate, amylopectin, alpha-amylose, ascorbic acid, ascorbyl palmitate, aspartame, bacteriostatic water for injection, bentonite, bentonite magma, benzalkonium chloride, benzethonium chloride, benzoic acid, benzyl alcohol, benzyl benzoate, bronopol, butylated hydroxyanisole, butylated hydroxytoluene, butylparaben, butylparaben sodium, calcium alginate, calcium ascorbate, calcium carbonate, calcium cyclamate, dibasic anhydrous calcium phosphate, dibasic dehydrate calcium phosphate, tribasic calcium phosphate, calcium propionate, calcium silicate, calcium sorbate, calcium stearate, calcium sulfate, calcium sulfate hemihydrate, canola oil, carbomer, carbon dioxide, carboxymethyl cellulose calcium, carboxymethyl cellulose sodium, beta-carotene, carrageenan, castor oil, hydrogenated castor oil, cationic emulsifying wax, cellulose acetate, cellulose acetate phthalate, ethyl cellulose, microcrystalline cellulose, powdered cellulose, silicified microcrystalline cellulose, sodium carboxymethyl cellulose, cetostearyl alcohol, cetrimide, cetyl alcohol, chlorhexidine, chlorobutanol, chlorocresol, cholesterol, chlorhexidine acetate, chlorhexidine gluconate, chlorhexidine hydrochloride, chlorodifluoroethane (HCFC), chlorodifluoromethane, chlorofluorocarbons (CFC)chlorophenoxyethanol, chloroxylenol, corn syrup solids, anhydrous citric acid, citric acid monohydrate, cocoa butter, coloring agents, corn oil, cottonseed oil, cresol, m-cresol, o-cresol, p-cresol, croscarmellose sodium, crospovidone, cyclamic acid, cyclodextrins, dextrates, dextrin, dextrose, dextrose anhydrous, diazolidinyl urea, dibutyl phthalate, dibutyl sebacate, diethanolamine, diethyl phthalate, difluoroethane (HFC), dimethyl-beta-cyclodextrin, cyclodextrin-type compounds such as Captisol®, dimethyl ether, dimethyl phthalate, dipotassium edentate, disodium edentate, disodium hydrogen phosphate, docusate calcium, docusate potassium, docusate sodium, dodecyl gallate, dodecyltrimethylammonium bromide, edentate calcium disodium, edtic acid, eglumine, ethyl alcohol, ethylcellulose, ethyl gallate, ethyl laurate, ethyl maltol, ethyl oleate, ethylparaben, ethylparaben potassium, ethylparaben sodium, ethyl vanillin, fructose, fructose liquid, fructose milled, fructose pyrogen-free, powdered fructose, fumaric acid, gelatin, glucose, liquid glucose, glyceride mixtures of saturated vegetable fatty acids, glycerin, glyceryl behenate, glyceryl monooleate, glyceryl monostearate, self-emulsifying glyceryl monostearate, glyceryl palmitostearate, glycine, glycols, glycofurol, guar gum, heptafluoropropane (HFC), hexadecyltrimethylammonium bromide, high fructose syrup, human serum albumin, hydrocarbons (HC), dilute hydrochloric acid, hydrogenated vegetable oil, type II, hydroxyethyl cellulose, 2-hydroxyethyl-beta-cyclodextrin, hydroxypropyl cellulose, low-substituted hydroxypropyl cellulose, 2-hydroxypropyl-beta-cyclodextrin, hydroxypropyl methylcellulose, hydroxypropyl methylcellulose phthalate, imidurea, indigo carmine, ion exchangers, iron oxides, isopropyl alcohol, isopropyl myristate, isopropyl palmitate, isotonic saline, kaolin, lactic acid, lactitol, lactose, lanolin, lanolin alcohols, anhydrous lanolin, lecithin, magnesium aluminum silicate, magnesium carbonate, normal magnesium carbonate, magnesium carbonate anhydrous, magnesium carbonate hydroxide, magnesium hydroxide, magnesium lauryl sulfate, magnesium oxide, magnesium silicate, magnesium stearate, magnesium trisilicate, magnesium trisilicate anhydrous, malic acid, malt, maltitol, maltitol solution, maltodextrin, maltol, maltose, mannitol, medium chain triglycerides, meglumine, menthol, methylcellulose, methyl methacrylate, methyl oleate, methylparaben, methylparaben potassium, methylparaben sodium, microcrystalline cellulose and carboxymethylcellulose sodium, mineral oil, light mineral oil, mineral oil and lanolin alcohols, oil, olive oil, monoethanolamine, montmorillonite, octyl gallate, oleic acid, palmitic acid, paraffin, peanut oil, petrolatum, petrolatum and lanolin alcohols, pharmaceutical glaze, phenol, liquified phenol, phenoxyethanol, phenoxypropanol, phenylethyl alcohol, phenylmercuric acetate, phenylmercuric borate, phenylmercuric nitrate, polacrilin, polacrilin potassium, poloxamer, polydextrose, polyethylene glycol, polyethylene oxide, polyacrylates, polyethylene-polyoxypropylene-block polymers, polymethacrylates, polyoxyethylene alkyl ethers, polyoxyethylene castor oil derivatives, polyoxyethylene sorbitol fatty acid esters, polyoxyethylene stearates, polyvinyl alcohol, polyvinyl pyrrolidone, potassium alginate, potassium benzoate, potassium bicarbonate, potassium bisulfite, potassium chloride, potassium citrate, potassium citrate anhydrous, potassium hydrogen phosphate, potassium metabisulfite, monobasic potassium phosphate, potassium propionate, potassium sorbate, povidone, propanol, propionic acid, propylene carbonate, propylene glycol, propylene glycol alginate, propyl gallate, propylparaben, propylparaben potassium, propylparaben sodium, protamine sulfate, rapeseed oil, Ringer's solution, saccharin, saccharin ammonium, saccharin calcium, saccharin sodium, safflower oil, saponite, serum proteins, sesame oil, colloidal silica, colloidal silicon dioxide, sodium alginate, sodium ascorbate, sodium benzoate, sodium bicarbonate, sodium bisulfite, sodium chloride, anhydrous sodium citrate, sodium citrate dehydrate, sodium chloride, sodium cyclamate, sodium edentate, sodium dodecyl sulfate, sodium lauryl sulfate, sodium metabisulfite, sodium phosphate, dibasic, sodium phosphate, monobasic, sodium phosphate, tribasic, anhydrous sodium propionate, sodium propionate, sodium sorbate, sodium starch glycolate, sodium stearyl fumarate, sodium sulfite, sorbic acid, sorbitan esters (sorbitan fatty esters), sorbitol, sorbitol solution 70%, soybean oil, spermaceti wax, starch, corn starch, potato starch, pregelatinized starch, sterilizable maize starch, stearic acid, purified stearic acid, stearyl alcohol, sucrose, sugars, compressible sugar, confectioner's sugar, sugar spheres, invert sugar, Sugartab, Sunset Yellow FCF, synthetic paraffin, talc, tartaric acid, tartrazine, tetrafluoroethane (HFC), theobroma oil, thimerosal, titanium dioxide, alpha tocopherol, tocopheryl acetate, alpha tocopheryl acid succinate, beta-tocopherol, delta-tocopherol, gamma-tocopherol, tragacanth, triacetin, tributyl citrate, triethanolamine, triethyl citrate, trimethyl-beta-cyclodextrin, trimethyltetradecylammonium bromide, tris buffer, trisodium edentate, vanillin, type I hydrogenated vegetable oil, water, soft water, hard water, carbon dioxide-free water, pyrogen-free water, water for injection, sterile water for inhalation, sterile water for injection, sterile water for irrigation, waxes, anionic emulsifying wax, carnauba wax, cationic emulsifying wax, cetyl ester wax, microcrystalline wax, nonionic emulsifying wax, suppository wax, white wax, yellow wax, white petrolatum, wool fat, xanthan gum, xylitol, zein, zinc propionate, zinc salts, zinc stearate, or any excipient in the Handbook of Pharmaceutical Excipients, Third Edition, A. H. Kibbe (Pharmaceutical Press, London, UK, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety, discloses various components used in formulating pharmaceutically acceptable compositions and known techniques for the preparation thereof. Except insofar as any conventional agent is incompatible with the pharmaceutical compositions, its use in pharmaceutical compositions is contemplated. Supplementary active ingredients also can be incorporated into the compositions.

In some embodiments, the foregoing component(s) may be present in the pharmaceutical composition at any concentration, such as, for example, at least A, wherein A is 0.0001% w/v, 0.001% w/v, 0.01% w/v, 0.1% w/v, 1% w/v, 2% w/v, 5% w/v, 10% w/v, 20% w/v, 30% w/v, 40% w/v, 50% w/v, 60% w/v, 70% w/v, 80% w/v, or 90% w/v. In some embodiments, the foregoing component(s) may be present in the pharmaceutical composition at any concentration, such as, for example, at most B, wherein B is 90% w/v, 80% w/v, 70% w/v, 60% w/v, 50% w/v, 40% w/v, 30% w/v, 20% w/v, 10% w/v, 5% w/v, 2% w/v, 1% w/v, 0.1% w/v, 0.001% w/v, or 0.0001%. In other embodiments, the foregoing component(s) may be present in the pharmaceutical composition at any concentration range, such as, for example from about A to about B. In some embodiments, A is 0.0001% and B is 90%.

The pharmaceutical compositions may be formulated to achieve a physiologically compatible pH. In some embodiments, the pH of the pharmaceutical composition may be at least 5, at least 5.5, at least 6, at least 6.5, at least 7, at least 7.5, at least 8, at least 8.5, at least 9, at least 9.5, at least 10, or at least 10.5 up to and including pH 11, depending on the formulation and route of administration. In certain embodiments, the pharmaceutical compositions may comprise buffering agents to achieve a physiological compatible pH. The buffering agents may include any compounds capable of buffering at the desired pH such as, for example, phosphate buffers (e.g., PBS), triethanolamine, Tris, bicine, TAPS, tricine, HEPES, TES, MOPS, PIPES, cacodylate, MES, and others. In certain embodiments, the strength of the buffer is at least 0.5 mM, at least 1 mM, at least 5 mM, at least 10 mM, at least 20 mM, at least 30 mM, at least 40 mM, at least 50 mM, at least 60 mM, at least 70 mM, at least 80 mM, at least 90 mM, at least 100 mM, at least 120 mM, at least 150 mM, or at least 200 mM. In some embodiments, the strength of the buffer is no more than 300 mM (e.g., at most 200 mM, at most 100 mM, at most 90 mM, at most 80 mM, at most 70 mM, at most 60 mM, at most 50 mM, at most 40 mM, at most 30 mM, at most 20 mM, at most 10 mM, at most 5 mM, at most 1 mM).

### Routes of Administration

The following discussion on routes of administration is provided to illustrate exemplary embodiments and should not be construed as limiting the scope in any way.

Formulations suitable for oral administration can consist of (a) liquid solutions, such as an effective amount of the isolated Angptl-FLD polypeptide of the invention dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the isolated Angptl-FLD polypeptide of the invention in a flavor, usually sucrose and acacia or tragacanth, as well as pastilles comprising the peptide of the present invention in an inert base, such as gelatin and glycerin, or sucrose and acacia, emulsions, gels, and the like containing, in addition to, such excipients as are known in the art.

The isolated Angptl4-FLD polypeptideof the invention, alone or in combination with other suitable components, can be delivered via pulmonary administration and can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa. In some embodiments, the isolated Angptl4-FLD polypeptide is formulated into a powder blend or into microparticles or nanoparticles. Suitable pulmonary formulations are known in the art. See, e.g., Qian et al., Int J Pharm 366: 218-220 (2009); Adjei and Garren, Pharmaceutical Research, 7(6): 565-569 (1990); Kawashima et al., J Controlled Release 62(1-2): 279-287 (1999); Liu et al., Pharm Res 10(2): 228-232 (1993); International Patent Application Publication Nos. WO 2007/133747 and WO 2007/141411.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. The isolated Angptl-FLD polypeptide of the invention can be administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-153-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-beta-aminopropionates, and 2-alkyl-imidazoline quaternary ammonium salts, and (e) mixtures thereof.

The parenteral formulations will typically contain from about 0.5% to about 25% by weight of the peptide of the isolated Angptl-FLD polypeptide of the invention in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The present invention also provides injectable formulations. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

Additionally, the isolated Angptl-FLD polypeptide of the invention can be made into suppositories for rectal administration by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

It will be appreciated by one of skill in the art that, in addition to the above-described pharmaceutical compositions, the isolated Angptl4-FLD polypeptide of the invention can be formulated as inclusion complexes, such as cyclodextrin inclusion complexes, or liposomes.

### Dose

The compositions of the present invention comprising isolated Angptl4-FLD polypeptide, as described herein are believed to be useful in methods of treating a disease or medical condition in which the isolated Angptl4-FLD polypeptide plays a role. For purposes of the present invention, the amount or dose of the composition of the present invention administered should be sufficient to effect, e.g., a therapeutic or prophylactic response, in the subject or animal over a reasonable time frame. For example, in exemplary embodiments, the dose of the composition of the present invention is sufficient to stimulate cAMP secretion from cells as described herein or sufficient to decrease blood glucose levels, fat levels, food intake levels, or body weight of a mammal, in a period of from about 1 to 4 minutes, 1 to 4 hours or 1 to 4 weeks or longer, e.g., 5 to 20 or more weeks, from the time of administration. In certain embodiments, the time period could be even longer. The dose will be determined by the efficacy of the particular composition of the present invention and the condition of the subject (e.g., human), as well as the body weight of the subject (e.g., human) to be treated.

Many assays for determining an administered dose are known in the art. For purposes herein, an assay, which comprises comparing the extent to which blood glucose levels or body weight are lowered upon administration of a given dose of the composition of the present invention to a mammal among a set of mammals of which is each given a different dose of the composition, could be used to determine a starting dose to be administered to a mammal. The extent to which blood glucose levels or body weight are lowered upon administration of a certain dose can be assayed by methods known in the art, including, for instance, the methods described in the Examples.

Typically, the attending physician will decide the dosage of the composition of the present invention with which to treat each individual subject, taking into consideration a variety of factors, such as age, body weight, general health, diet, sex, composition of the present invention to be administered, route of administration, severity of the condition being treated, and clinical effect to be achieved. The dose of the composition of the present invention also will be determined by the existence, nature and extent of any adverse side effects that might accompany the administration of a particular peptide of the present invention.

### Controlled Release Formulations and Time of Administration

Alternatively, the peptides described herein can be modified into a depot form, such that the manner in which the isolated Angptl-FLD polypeptide of the invention is released into the body to which it is administered is controlled with respect to time and location within the body (see, for example, U.S. Pat. No. 4,450,150). Depot forms of isolated Angptl4-FLD polypeptide can be, for example, an implantable composition comprising the peptide of the present invention and a porous or non-porous material, such as a polymer, wherein the peptide of the present invention is encapsulated by or diffused throughout the material and/or degradation of the non-porous material. The depot is then implanted into the desired location within the body and the isolated Angptl4-FLD polypeptide is released from the implant at a predetermined rate.

The pharmaceutical composition in certain aspects is modified to have any type of in vivo release profile. In some aspects, the pharmaceutical composition is an immediate release, controlled release, sustained release, extended release, delayed release, or bi-phasic release formulation. Methods of formulating peptides for controlled release are known in the art. See, for example, Qian et al., J Pharm 374: 46-52 (2009) and International Patent Application Publication Nos. WO 2008/130158, WO2004/033036; WO2000/032218; and WO 1999/040942.

The instant compositions may further comprise, for example, micelles or liposomes, or some other encapsulated form, or may be administered in an extended release form to provide a prolonged storage and/or delivery effect.

The disclosed pharmaceutical formulations may be administered according to any regime including, for example, daily (1 time per day, 2 times per day, 3 times per day, 4 times per day, 5 times per day, 6 times per day), every two days, every three days, every four days, every five days, every six days, weekly, bi-weekly, every three weeks, monthly, or bi-monthly.

### Combinations

The isolated Angptl4-FLD polypeptide described herein may be administered alone or in combination with other therapeutic agents which aim to treat or prevent any of the diseases or medical conditions described herein. For example, the peptides described herein may be co-administered with (simultaneously or sequentially) an anti-diabetic or anti-obesity agent. Anti-diabetic agents known in the art or under investigation include insulin, leptin, Peptide YY (PYY), Pancreatic Peptide (PP), fibroblast growth factor 21 (FGF21), Y2Y4 receptor agonists, sulfonylureas, such as tolbutamide (Orinase), acetohexamide (Dymelor), tolazamide (Tolinase), chlorpropamide (Diabinese), glipizide (Glucotrol), glyburide (Diabeta, Micronase, Glynase), glimepiride (Amaryl), or gliclazide (Diamicron); meglitinides, such as repaglinide (Prandin) or nateglinide (Starlix); biguanides such as metformin (Glucophage) or phenformin; thiazolidinediones such as rosiglitazone (Avandia), pioglitazone (Actos), or troglitazone (Rezulin), or other PPAR.gamma. inhibitors; alpha glucosidase inhibitors that inhibit carbohydrate digestion, such as miglitol (Glyset), acarbose (Precose/Glucobay); exenatide (Byetta) or pramlintide; Dipeptidyl peptidase-4 (DPP-4) inhibitors such as vildagliptin or sitagliptin; SGLT (sodium-dependent glucose transporter 1) inhibitors; glucokinase activators (GKA); glucagon receptor antagonists (GRA); or FBPase (fructose 1,6-bisphosphatase) inhibitors.

Anti-obesity agents known in the art or under investigation include appetite suppressants, including phenethylamine type stimulants, phentermine (optionally with fenfluramine or dexfenfluramine), diethylpropion (Tenuate®), phendimetrazine (Prelu-2®, Bontril®), benzphetamine (Didrex®), sibutramine (Meridia®, Reductil®); rimonabant (Acomplia®), other cannabinoid receptor antagonists; oxyntomodulin; fluoxetine hydrochloride (Prozac); Qnexa (topiramate and phentermine), Excalia (bupropion and zonisamide) or Contrave (bupropion and naltrexone); or lipase inhibitors, similar to XENICAL (Orlistat) or Cetilistat (also known as ATL-962), or GT 389-255.

The isolated Angptl-FLD polypeptide of the invention, in some embodiments, are co-administered with an agent for treatment of non-alcoholic fatty liver disease or NASH. Agents used to treat non-alcoholic fatty liver disease include ursodeoxycholic acid (a.k.a., Actigall, URSO, and Ursodiol), Metformin (Glucophage), rosiglitazone (Avandia), Clofibrate, Gemfibrozil, Polymixin B, and Betaine.

The isolated Angptl-FLD polypeptide of the invention, in some embodiments are co-administered with an agent for treatment of a neurodegenerative disease, e.g., Parkinson's Disease. Anti-Parkinson's Disease agents are furthermore known in the art and include, but not limited to, levodopa, carbidopa, anticholinergics, bromocriptine, pramipexole, and ropinirole, amantadine, and rasagiline.

In view of the foregoing, the present invention further provide pharmaceutical compositions and kits additionally comprising one of these other therapeutic agents. The additional therapeutic agent may be administered simultaneously or sequentially with the peptide of the present invention. In some aspects, the peptide is administered before the additional therapeutic agent, while in some embodiments, the isolated Angptl4-FLD polypeptide is administered after the additional therapeutic agent.

### Uses

Based on the information provided for the first time herein, it is contemplated that the compositions (e.g., related pharmaceutical compositions) of the present invention are useful for treatment of a disease or medical condition. The method comprises providing to the subject a composition or conjugate in accordance with any of those described herein in an amount effective to treat or prevent the disease or medical condition.

In some embodiments, the disease or medical condition is metabolic syndrome. Metabolic Syndrome, also known as metabolic syndrome X, insulin resistance syndrome or Reaven's syndrome, is a disorder that affects over 50 million Americans. Metabolic Syndrome is typically characterized by a clustering of at least three or more of the following risk factors: (1) abdominal obesity (excessive fat tissue in and around the abdomen), (2) atherogenic dyslipidemia (blood fat disorders including high triglycerides, low HDL cholesterol and high LDL cholesterol that enhance the accumulation of plaque in the artery walls), (3) elevated blood pressure, (4) insulin resistance or glucose intolerance, (5) prothrombotic state (e.g., high fibrinogen or plasminogen activator inhibitor-1 in blood), and (6) pro-inflammatory state (e.g., elevated C-reactive protein in blood). Other risk factors may include aging, hormonal imbalance and genetic predisposition.

Metabolic Syndrome is associated with an increased the risk of coronary heart disease and other disorders related to the accumulation of vascular plaque, such as stroke and peripheral vascular disease, referred to as atherosclerotic cardiovascular disease (ASCVD). Subjects with Metabolic Syndrome may progress from an insulin resistant state in its early stages to full blown type II diabetes with further increasing risk of ASCVD. Without intending to be bound by any particular theory, the relationship between insulin resistance, Metabolic Syndrome and vascular disease may involve one or more concurrent pathogenic mechanisms including impaired insulin-stimulated vasodilation, insulin resistance-associated reduction in NO availability due to enhanced oxidative stress, and abnormalities in adipocyte-drived hormones such as adiponectin (Lteif and Mather, Can. J. Cardiol. 20 (suppl. B):66B-76B (2004)).

According to the 2001 National Cholesterol Education Program Adult Treatment Panel (ATP III), any three of the following traits in the same individual meet the criteria for Metabolic Syndrome: (a) abdominal obesity (a waist circumference over 102 cm in men and over 88 cm in women); (b) serum triglycerides (150 mg/dl or above); (c) HDL cholesterol (40 mg/dl or lower in men and 50 mg/dl or lower in women); (d) blood pressure (130/85 or more); and (e) fasting blood glucose (110 mg/dl or above). According to the World Health Organization (WHO), an individual having high insulin levels (an elevated fasting blood glucose or an elevated post meal glucose alone) with at least two of the following criteria meets the criteria for Metabolic Syndrome: (a) abdominal obesity (waist to hip ratio of greater than 0.9, a body mass index of at least 30 kg/m2, or a waist measurement over 37 inches); (b) cholesterol panel showing a triglyceride level of at least 150 mg/dl or an HDL cholesterol lower than 35 mg/dl; (c) blood pressure of 140/90 or more, or on treatment for high blood pressure). (Mathur, Ruchi, “Metabolic Syndrome,” ed. Shiel, Jr., William C., MedicineNet.com, May 11, 2009).

For purposes herein, if an individual meets the criteria of either or both of the criteria set forth by the 2001 National Cholesterol Education Program Adult Treatment Panel or the WHO, that individual is considered as afflicted with Metabolic Syndrome.

Without being bound to any particular theory, compositions and conjugates described herein are useful for treating Metabolic Syndrome. Accordingly, the invention provides a method of preventing or treating Metabolic Syndrome, or reducing one, two, three or more risk factors thereof, in a subject, comprising providing to the subject a composition described herein in an amount effective to prevent or treat Metabolic Syndrome, or the risk factor thereof.

In some embodiments, the method treats a hyperglycemic medical condition. In certain aspects, the hyperglycemic medical condition is diabetes, diabetes mellitus type I, diabetes mellitus type II, or gestational diabetes, either insulin-dependent or non-insulin-dependent. In some aspects, the method treats the hyperglycemic medical condition by reducing one or more complications of diabetes including nephropathy, retinopathy and vascular disease.

In some aspects, the disease or medical condition is obesity. In some aspects, the obesity is drug-induced obesity. In some aspects, the method treats obesity by preventing or reducing weight gain or increasing weight loss in the subject. In some aspects, the method treats obesity by reducing appetite, decreasing food intake, lowering the levels of fat in the subject, or decreasing the rate of movement of food through the gastrointestinal system.

Because obesity is associated with the onset or progression of other diseases, the methods of treating obesity are further useful in methods of reducing complications associated with obesity including vascular disease (coronary artery disease, stroke, peripheral vascular disease, ischemia reperfusion, etc.), hypertension, onset of diabetes type II, hyperlipidemia and musculoskeletal diseases. The present invention, in various embodiments, accordingly provides methods of treating or preventing these obesity-associated complications.

In some embodiments, the disease or medical condition is Nonalcoholic fatty liver disease (NAFLD). NAFLD refers to a wide spectrum of liver disease ranging from simple fatty liver (steatosis), to nonalcoholic steatohepatitis (NASH), to cirrhosis (irreversible, advanced scarring of the liver). All of the stages of NAFLD have in common the accumulation of fat (fatty infiltration) in the liver cells (hepatocytes). Simple fatty liver is the abnormal accumulation of a certain type of fat, triglyceride, in the liver cells with no inflammation or scarring. In NASH, the fat accumulation is associated with varying degrees of inflammation (hepatitis) and scarring (fibrosis) of the liver. The inflammatory cells can destroy the liver cells (hepatocellular necrosis). In the terms “steatohepatitis” and “steatonecrosis”, steato refers to fatty infiltration, hepatitis refers to inflammation in the liver, and necrosis refers to destroyed liver cells. NASH can ultimately lead to scarring of the liver (fibrosis) and then irreversible, advanced scarring (cirrhosis). Cirrhosis that is caused by NASH is the last and most severe stage in the NAFLD spectrum. (Mendler, Michel, “Fatty Liver: Nonalcoholic Fatty Liver Disease (NAFLD) and Nonalcoholic Steatohepatitis (NASH),” ed. Schoenfield, Leslie J., MedicineNet.com, Aug. 29, 2005).

Alcoholic Liver Disease, or Alcohol-Induced Liver Disease, encompasses three pathologically distinct liver diseases related to or caused by the excessive consumption of alcohol: fatty liver (steatosis), chronic or acute hepatitis, and cirrhosis. Alcoholic hepatitis can range from a mild hepatitis, with abnormal laboratory tests being the only indication of disease, to severe liver dysfunction with complications such as jaundice (yellow skin caused by bilirubin retention), hepatic encephalopathy (neurological dysfunction caused by liver failure), ascites (fluid accumulation in the abdomen), bleeding esophageal varices (varicose veins in the esophagus), abnormal blood clotting and coma. Histologically, alcoholic hepatitis has a characteristic appearance with ballooning degeneration of hepatocytes, inflammation with neutrophils and sometimes Mallory bodies (abnormal aggregations of cellular intermediate filament proteins). Cirrhosis is characterized anatomically by widespread nodules in the liver combined with fibrosis. (Worman, Howard J., “Alcoholic Liver Disease”, Columbia University Medical Center website).

Without being bound to any particular theory, the compositions and conjugates described herein are useful for the treatment of Alcoholic Liver Disease, NAFLD, or any stage thereof, including, for example, steatosis, steatohepatitis, hepatitis, hepatic inflammation, NASH, cirrhosis, or complications thereof. Accordingly, the present invention provides a method of preventing or treating Alcoholic Liver Disease, NAFLD, or any stage thereof, in a subject comprising providing to a subject a composition described herein in an amount effective to prevent or treat Alcoholic Liver Disease, NAFLD, or the stage thereof. Such treatment methods include reduction in one, two, three or more of the following: liver fat content, incidence or progression of cirrhosis, incidence of hepatocellular carcinoma, signs of inflammation, e.g., abnormal hepatic enzyme levels (e.g., aspartate aminotransferase AST and/or alanine aminotransferase ALT, or LDH), elevated serum ferritin, elevated serum bilirubin, and/or signs of fibrosis, e.g., elevated TGF-beta levels. In certain embodiments, the compositions are used treat subjects who have progressed beyond simple fatty liver (steatosis) and exhibit signs of inflammation or hepatitis. Such methods may result, for example, in reduction of AST and/or ALT levels.

Analogous to GLP-1 and exendin-4, which have been shown to have some neuroprotective effect, the isolated Angptl4-FLD polypeptide of the present invention also provides uses of the compositions described herein in treating neurodegenerative diseases, including but not limited to Alzheimer's disease, Parkinson's disease, Multiple Sclerosis, Amylotrophic Lateral Sclerosis, other demyelination related disorders, senile dementia, subcortical dementia, arteriosclerotic dementia, AIDS-associated dementia, or other dementias, a central nervous system cancer, traumatic brain injury, spinal cord injury, stroke or cerebral ischemia, cerebral vasculitis, epilepsy, Huntington's disease, Tourette's syndrome, Guillain Barre syndrome, Wilson disease, Pick's disease, neuroinflammatory disorders, encephalitis, encephalomyelitis or meningitis of viral, fungal or bacterial origin, or other central nervous system infections, prion diseases, cerebellar ataxias, cerebellar degeneration, spinocerebellar degeneration syndromes, Friedreichs ataxia, ataxia telangiectasia, spinal dysmyotrophy, progressive supranuclear palsy, dystonia, muscle spasticity, tremor, retinitis pigmentosa, striatonigral degeneration, mitochondrial encephalo-myopathies, neuronal ceroid lipofuscinosis, hepatic encephalopathies, renal encephalopathies, metabolic encephalopathies, toxin-induced encephalopathies, and radiation-induced brain damage.

In some embodiments, the compositions are used in conjunction with parenteral administration of nutrients to non-diabetic subjects in a hospital setting, e.g., to subjects receiving parenteral nutrition or total parenteral nutrition. Nonlimiting examples include surgery subjects, subjects in comas, subjects with digestive tract illness, or a nonfunctional gastrointestinal tract (e.g. due to surgical removal, blockage or impaired absorptive capacity, Crohn's disease, ulcerative colitis, gastrointestinal tract obstruction, gastrointestinal tract fistula, acute pancreatitis, ischemic bowel, major gastrointestinal surgery, certain congenital gastrointestinal tract anomalies, prolonged diarrhea, or short bowel syndrome due to surgery, subjects in shock, and subjects undergoing healing processes often receive parenteral administration of carbohydrates along with various combinations of lipids, electrolytes, minerals, vitamins and amino acids. The compositions comprising the isolated Angptl-FLD polypeptide of the invention, and the parenteral nutrition composition can be administered at the same time, at different times, before, or after each other, provided that the composition is exerting the desired biological effect at the time that the parenteral nutrition composition is being digested. For example, the parenteral nutrition may be administered, 1, 2 or 3 times per day, while the composition is administered once every other day, three times a week, two times a week, once a week, once every 2 weeks, once every 3 weeks, or once a month.

With regard to the above methods of treatment, the subject is any host. In some embodiments, the host is a mammal. As used herein, the term “mammal” refers to any vertebrate animal of the mammalia class, including, but not limited to, any of the monotreme, marsupial, and placental taxas. In some embodiments, the mammal is one of the mammals of the order Rodentia, such as mice and hamsters, and mammals of the order Logomorpha, such as rabbits. In certain embodiments, the mammals are from the order Carnivora, including Felines (cats) and Canines (dogs). In certain embodiments, the mammals are from the order Artiodactyla, including Bovines (cows) and S wines (pigs) or of the order Perssodactyla, including Equines (horses). In some instances, the mammals are of the order Primates, Ceboids, or Simoids (monkeys) or of the order Anthropoids (humans and apes). In particular embodiments, the mammal is a human.

### Kits

The present invention further provides kits comprising a peptide of the invention.

In some embodiments, the kit may include two separate containers, e.g., vials, tubes, bottles, single or multi-chambered pre-filled syringes, cartridges, infusion pumps (external or implantable), jet injectors, pre-filled pen devices and the like, one of which contains the isolated An tl-FLD polypeptide of the invention.

In some embodiments, the isolated Angptl-FLD polypeptide of the invention is provided in the kit as a lyophilized form or in an aqueous solution.

The kits in some embodiments comprise instructions for use. The instructions in some aspects include instructions for administration of the isolated An tl-FLD polypeptide of the invention.

In one embodiment the kit is provided with a device for administering the composition to a subject, e.g., syringe needle, pen device, jet injector or other needle-free injector. In accordance with one embodiment the device of the kit is an aerosol dispensing device, wherein the composition is prepackaged within the aerosol device. In another embodiment the kit comprises a syringe and a needle, and in one embodiment the sterile composition is prepackaged within the syringe.

In some embodiments, the kit comprises a pharmaceutically acceptable carrier, such as any of those described herein.

### EXAMPLES

Angiopoietin-like 4 (Angptl4) is a secreted protein that inhibits lipoprotein lipase (LPL) activity and promotes lipolysis in adipocytes. Previous studies have shown that the N-terminal coiledcoil domain of ANGPTL4 alone can inhibit LPL. Here, we report that the C-terminal fibrinogenlike domain (FLD) of ANGPTL4 alone can elevate intracellular cAMP levels and induce lipolysis in adipocytes. Therefore, the LPL inhibitory activity and the adipocyte lipolytic activity of ANGPTL4 can be separated. We overexpressed ANGPTL4 FLD with an adenoviral delivery system in mice to study its metabolic functions. We found that under chow diet, mice overexpressing LacZ (Ad-LacZ), full length ANGPTL4 (Ad-WT), or ANGPTL4 FLD (Ad-FLD) had similar food intake and body weight gain. Ad-WT mice showed increased plasma triglyceride (TG) levels, whereas Ad-FLD mice did not present such phenotype. Intriguingly, Ad-WT and Ad-FLD mice were markedly more glucose tolerant than Ad-LacZ mice. Under high-fat diet, all three groups of mice again had similar food intake. However, the adiposity of Ad-WT and Ad-FLD mice is significantly lower than in Ad-LacZ mice. Interestingly, Ad-FLD mice had improved glucose tolerance compared to Ad-WT and Ad-LacZ mice. Overall, our results suggest that overexpression of ANGPTL4 FLD not only protects mice from diet-induced obesity without affecting plasma TG levels, but also improves glucose homeostasis. Therefore, Angptl4 FLD is a potential therapeutic agent against metabolic diseases, such as obesity and type 2 diabetes.

### Example 1

FLD Induces Lipolysis and cAMP Levels in Mouse Primary Adipocytes

To test whether the LPL inhibitory effect of ANGPTL4 is required for its induction of lipolysis, we generated two ANGPTL4 mutants. An adenoviral vector expressing flag-tagged full length human ANGPTL4 (ANGPTL4) was provided from the laboratory of Ron Kahn (Joslin Diabetes Center). We then conducted site-directed mutagenesis to alter amino acid 40 from glutamic acid to lysine (E40K), which diminishes the LPL inhibitory activity of ANGPTL4 (18; 19; 39). We also performed a deletion to excise nucleotides encoding amino acid 38-165 of human ANGPTL4. This mutant (called as FLD), thus, lacked CCD. The first 37 amino acids were not deleted, as they contain signal peptide that allows FLD to be secreted. Adenoviruses expressing E40K, FLD, or ANGPTL4 (WT) were generated following manufacture's manual (AdEasy, Agilent). 293T cells were infected with these adenoviruses for 2-3 days, and E40K, FLD, or ANGPTL4 proteins were purified from media using flag affinity purification system (Sigma).

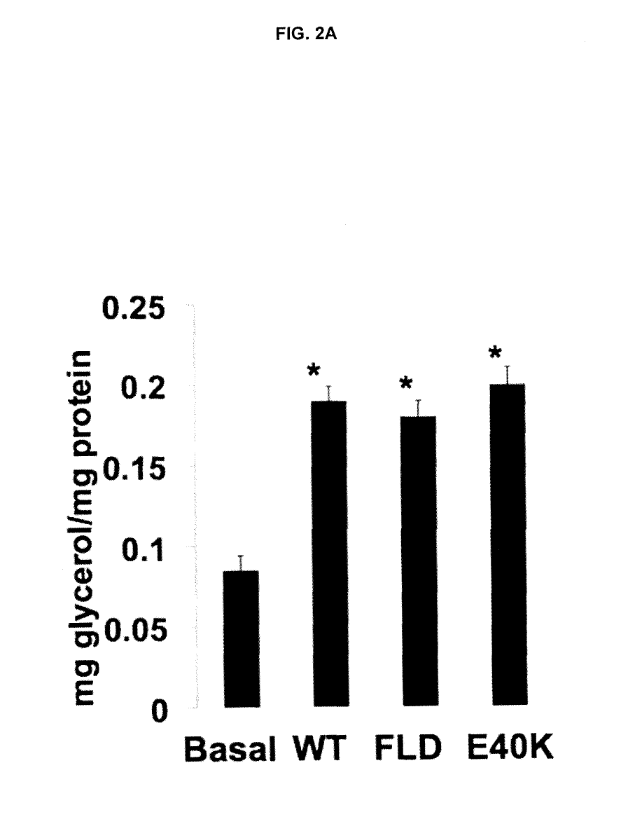

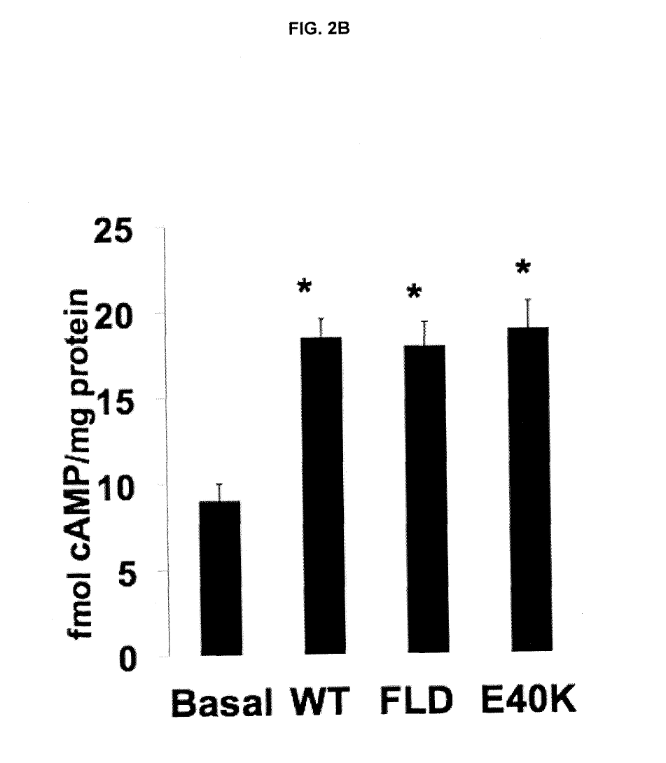

Our previous studies showed that Angptl4 concentration reached 0.9 mg/mg tissue (−18.3 nM) during normal state and 1.6 mg/mg tissue (−32.5 nM) upon fasting in the eWAT (12). In plasma, Angptl4 concentration is 2.4 nM during normal state and 11.6 nM upon fasting (12). Here we used Angptl4 concentration at normal state of WAT, 20 nM, for these preliminary experiments. We treated primary mouse adipocytes with 20 nM FLD, E40K or ANGPTL4 for 60 min and then measured glycerol levels in the media. We found that the levels of glycerol induced by FLD, E4OK and ANGPTL4 are similar (FIG. 2A). Moreover, the levels of intracellular cAMP induced by 30 min FLD, E40K, and ANGPTL4 treatment were also similar (FIG. 2B). These results indicated that the LPL inhibitory effect of

ANGPTL4 is not required to induce adipocyte lipolysis, and FLD alone is sufficient to activate cAMP signaling to promote lipolysis.

### Example 2

### Increasing Plasma FLD Levels Protected Mice From Diet-Induced Obesity

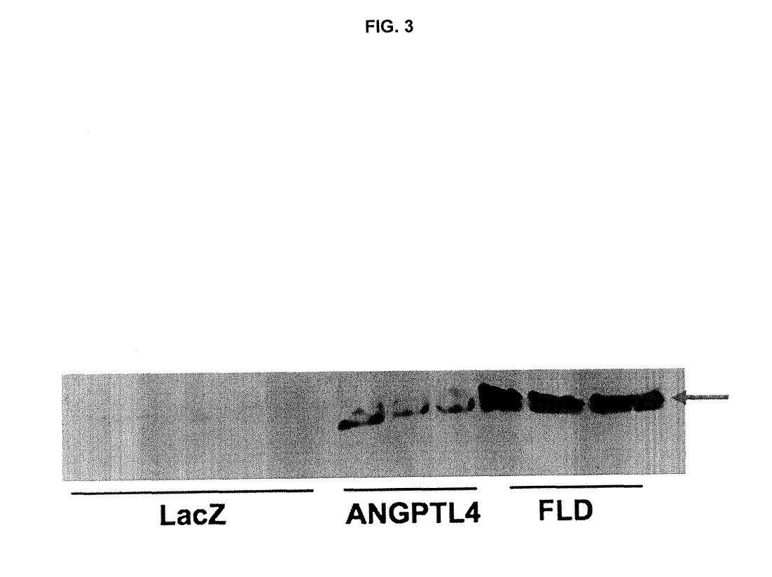

C57BL/6 mice were infected with adenovirus expressing FLD, ANGPTL4, or LacZ (control) and placed under high-fat diet (42% fat, Harlan Lab) for 3 weeks. A three-week time period was chosen, because adenoviral expression usually lasts 3-4 weeks. After 3 weeks, the expression of FLD and ANGPTL4 in plasma of adenovirus-infected mice was confirmed by immunoblots using flag antibody (FIG. 3). Because most ANGPTL4 produced in liver was processed to shorter forms, flag-tagged proteins detected in plasma of ANGPTL4- and FLD-expressing mice had similar molecular weight (FIG. 3). This phenomenon was reported from our and other laboratories (14-16). As Flag-tag was located at C-terminus, proteins detected in plasma of ANGPTL4-expressing mice were FLD. But N-terminal CCD should also be in circulation of these mice. Flag-tagged protein expression was lower in ANGPTL4-expressing mice. We will use higher titers of ANGPTL4-expressing virus in proposed experiments.

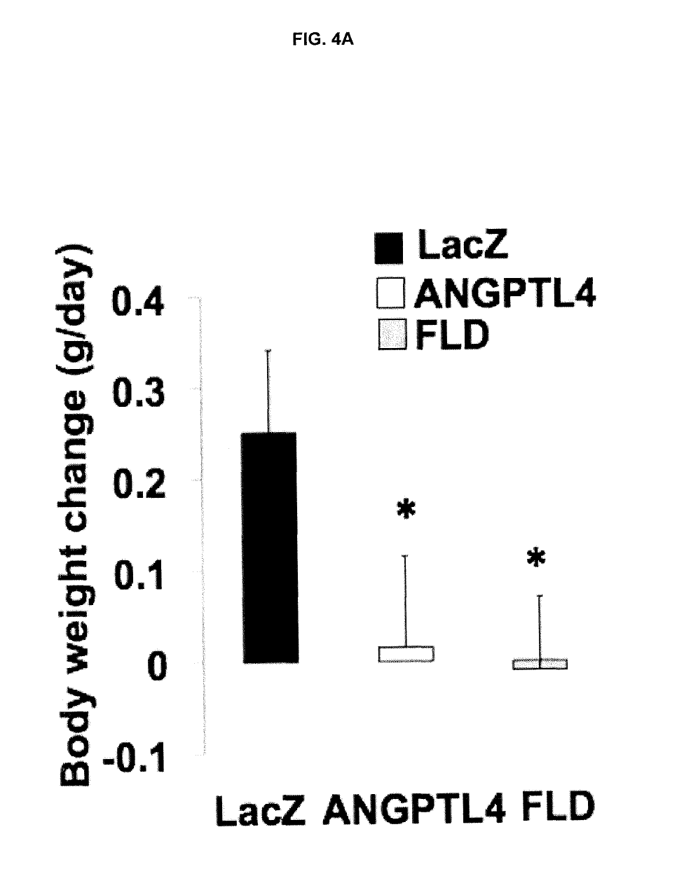

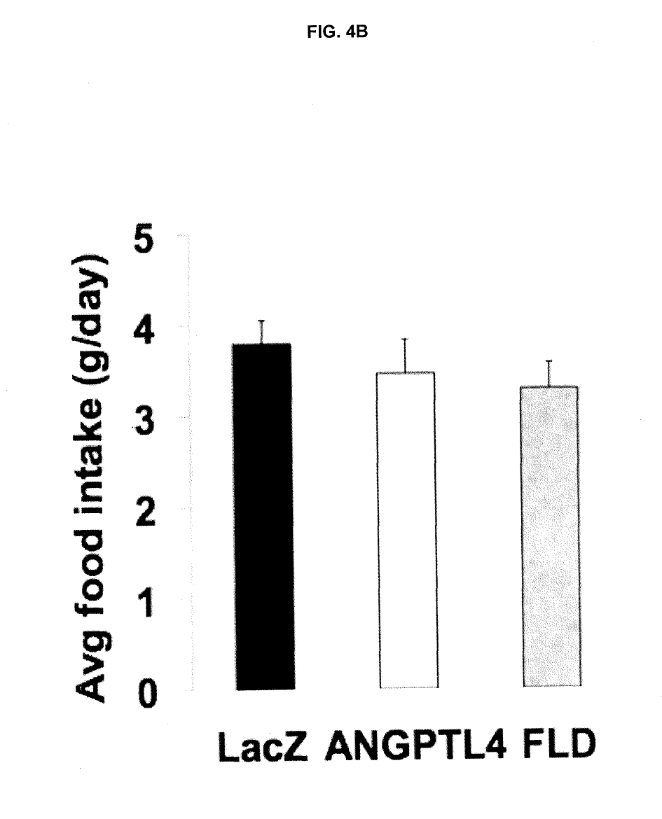

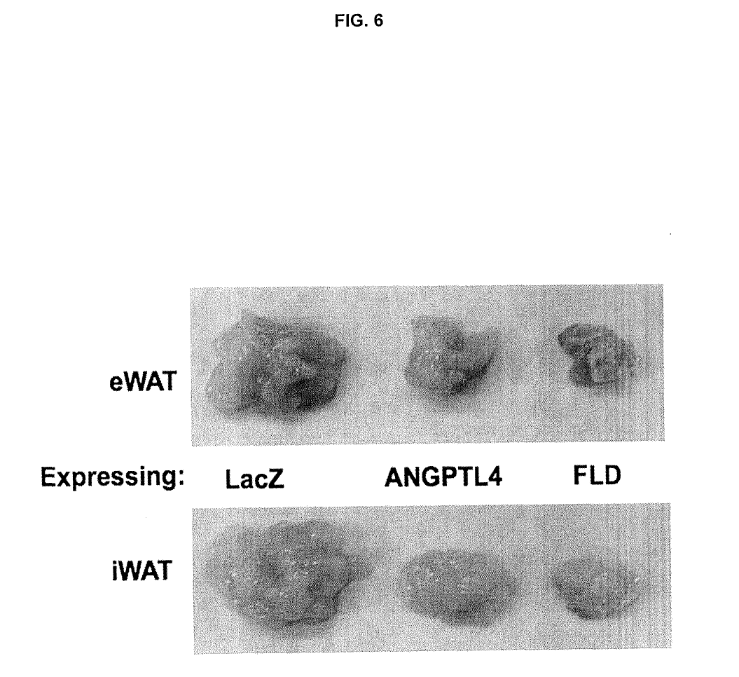

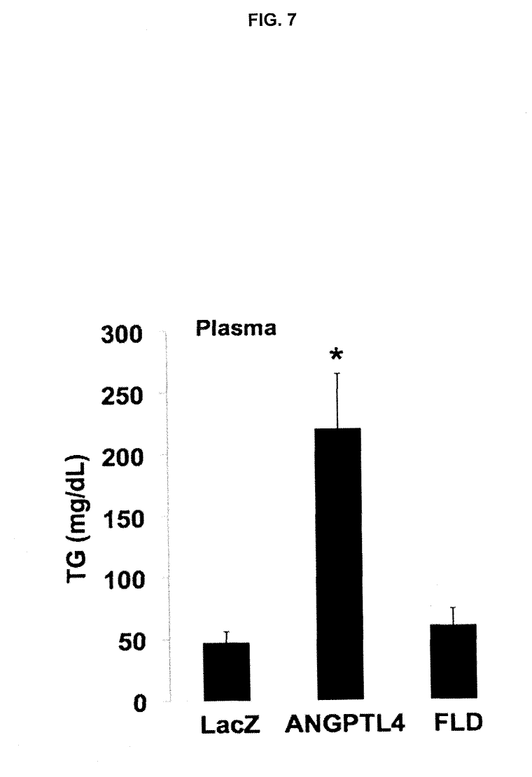

Under high-fat diet, mice expressing LacZ gained significant weight. However, weight gain for mice expressing ANGPTL4 or FLD was not significant (FIG. 4). Food intake was similar between these mice (FIG. 4). We found that the weights of iWAT, eWAT, and BAT were markedly lower in mice expressing ANGPTL4 or FLD (FIG. 6, BAT not shown), whereas the weights for other tissues were not statistically different (data not shown). Plasma TG levels in ANGPTL4-expressing mice were approximately four fold higher than mice expressing FLD or LacZ (FIG. 7). Overall, these results confirmed that FLD can protect mice from diet-induced obesity without affecting plasma TG levels.

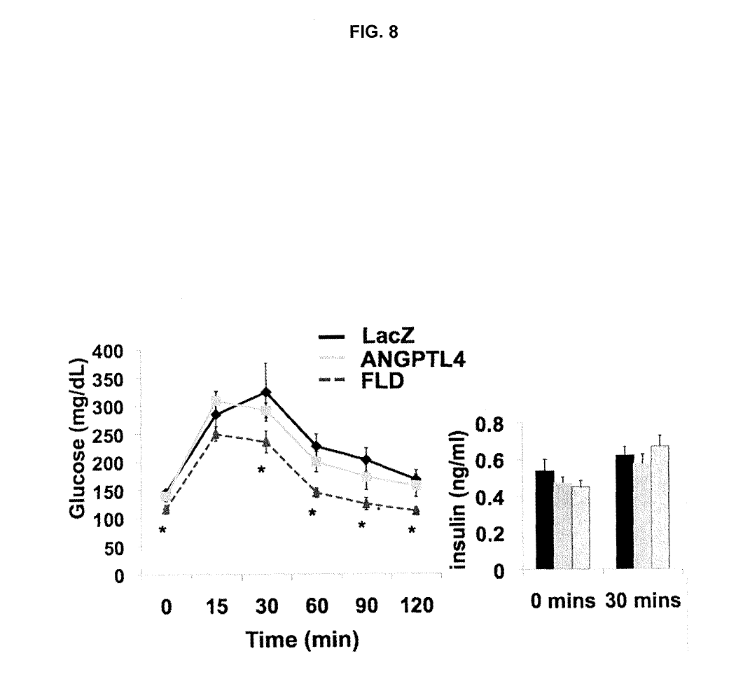

Glucose tolerance test (GTT) showed that FLD-expressing mice were more glucose tolerant than WT mice (FIG. 8), likely due to decreased adiposity. Surprisingly, glucose tolerance of ANGPTL4-expressing mice was not improved despite the reduction of adiposity (FIG. 8). It is possible that higher expression of ANGPTL4 is required to improve insulin sensitivity. Based on FIG. 3, adenovirus-mediated ANGPTL4 expression was lower than adenovirus-mediated FLD expression. Alternatively, increasing CCD levels may affect insulin sensitivity in ANGPTL4-expressing mice.

### Example 3

### The Expression of Fatty Acid Oxidative Genes was Increased in FLD-Overexpressing Mice

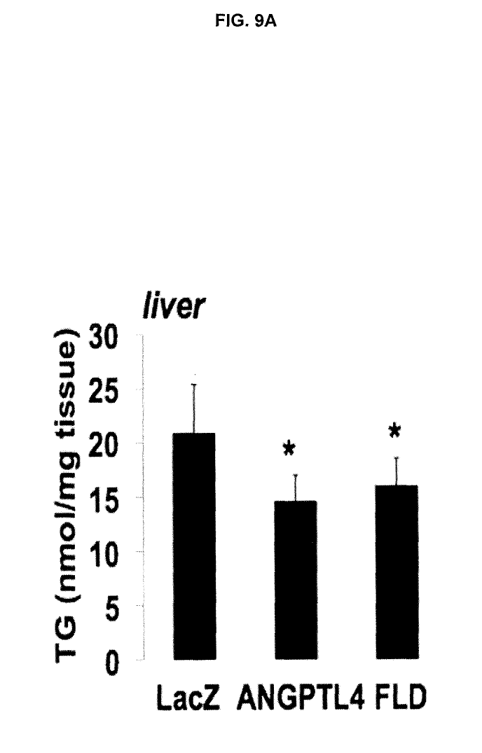

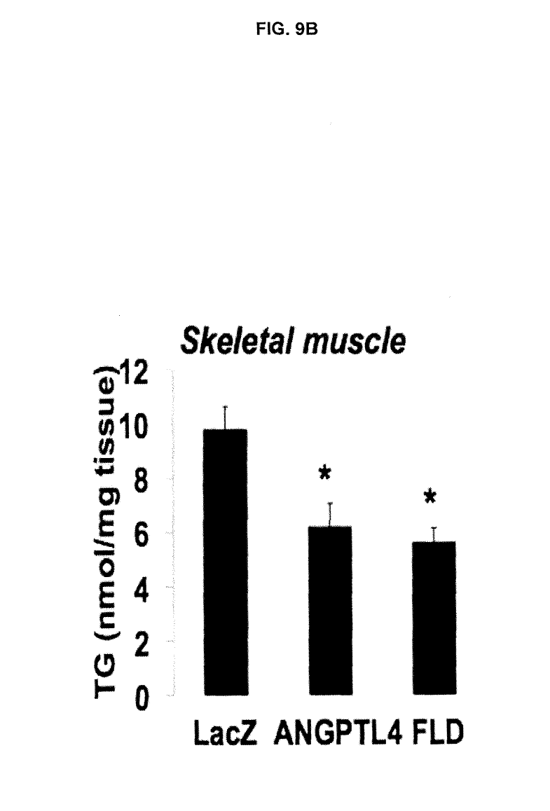

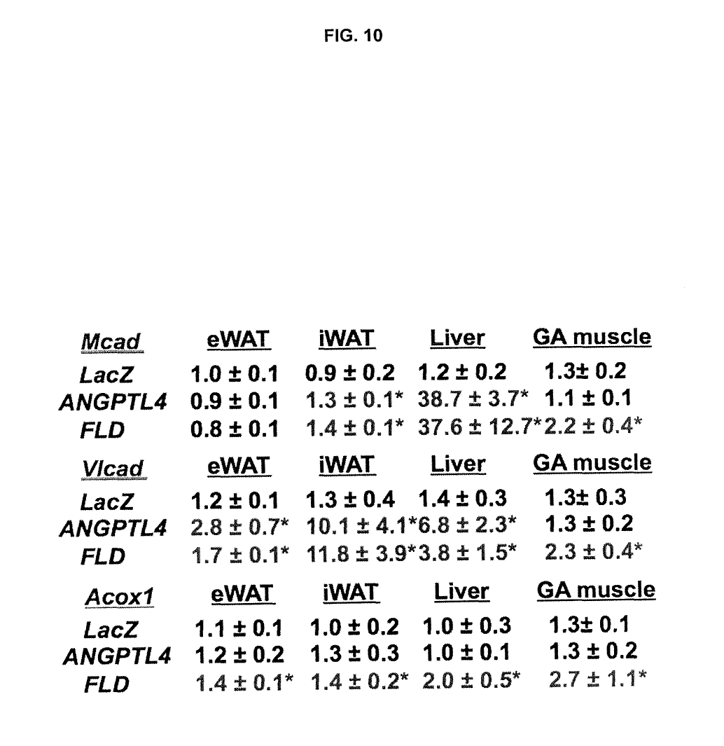

As tissue weight of iWAT, eWAT and BAT decreased markedly by FLD-induced lipolysis, FA generated from this process may be mobilized to other tissues. However, we found that TG levels in liver and gastrocnemius muscle were lower in mice overexpressing ANGPTL4 and FLD (FIG. 9A and FIG. 9B). Plasma NEFA levels were similar in LacZ-, FLD-, and ANGPTL4-expressing mice (data not shown). We reasoned that FA generated from lipolysis could be oxidized in WAT, liver, or skeletal muscle. We therefore examined the expression of fatty acid oxidative genes: very long chain acyl-coeznyme A dehydrogenase (Vlcad), medium chain acyl-coeznyme A dehydrogenase (Mcad), and acyl-coenzyme A oxidase 1 (Acoxl) in various tissues. These three genes were induced in almost all tissues tested in FLD-overexpressing mice (FIG. 10). In ANGPTL4-overexpressing mice, the expression of Mcad and Vlcad were increased in iWAT and liver (FIG. 10). We are currently examining the expression of more fatty acid oxidative genes.

### Example 4

Overexpression of FLD Improved Glucose and Pyruvate Tolerance in Mice Fed with Chow Diet

We also infected mice fed with chow diet with adenovirus expression FLD, ANGPTL4 or LacZ for 3 weeks. Food intake and weight gain were similar in these mice (data not shown). We did not observe any significant difference in TG levels in eWAT, iWAT, liver and skeletal muscle of mice overexpressing FLD, ANGPTL4 or LacZ (data not shown). The patterns of plasma TG levels were similar to those of high-fat diet experiments. Thus, FLD- and LacZ-overexpressing mice had similar plasma TG levels, whereas plasma TG levels were markedly higher in ANGPTL4-overexpressing mice (data not shown). We then measured plasma FFA levels. We found that plasma FFA levels were markedly higher in ANGPTL4- and FLD-overexpressing mice (4.7 and 3.9 mmole/L, respectively) than that of LacZ-ovexpressing mice (1.9 mmole/L, these results were from 5-6 mice for each treatment). These results were in agreement with lipolytic activity of FLD and ANGPTL4.

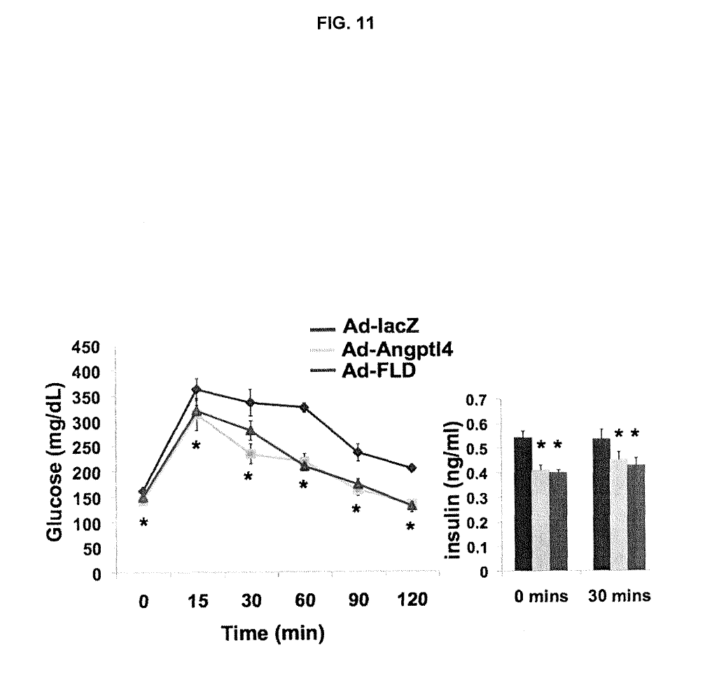

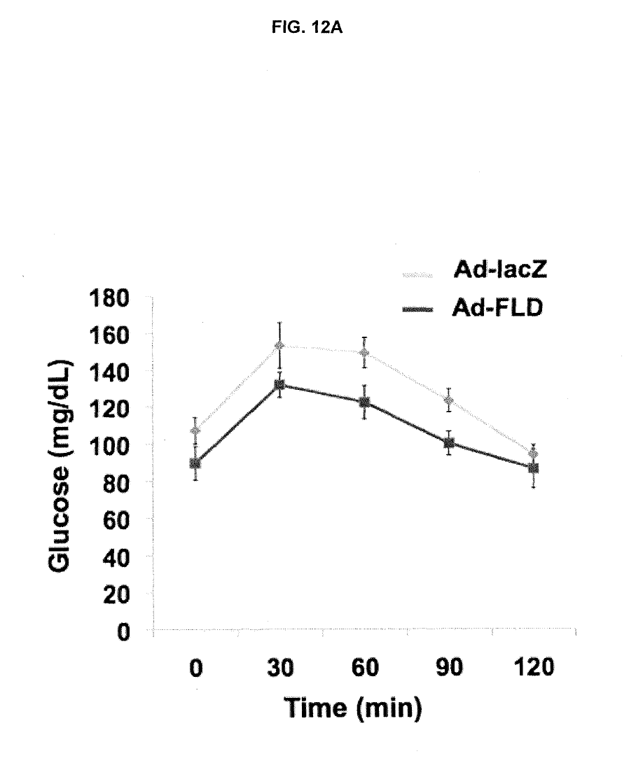

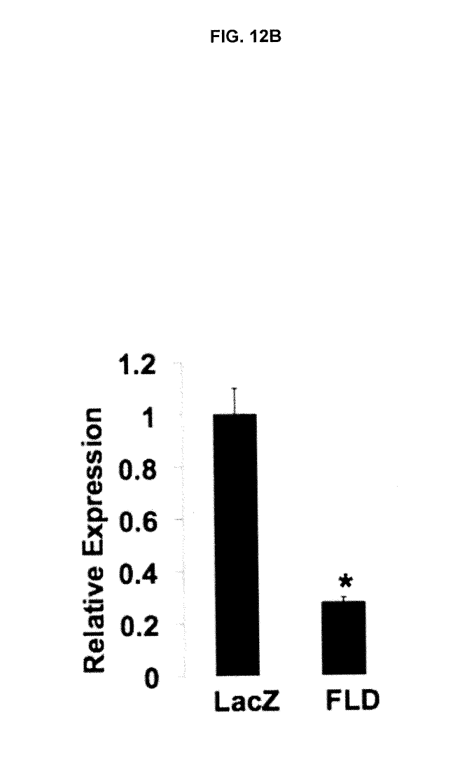

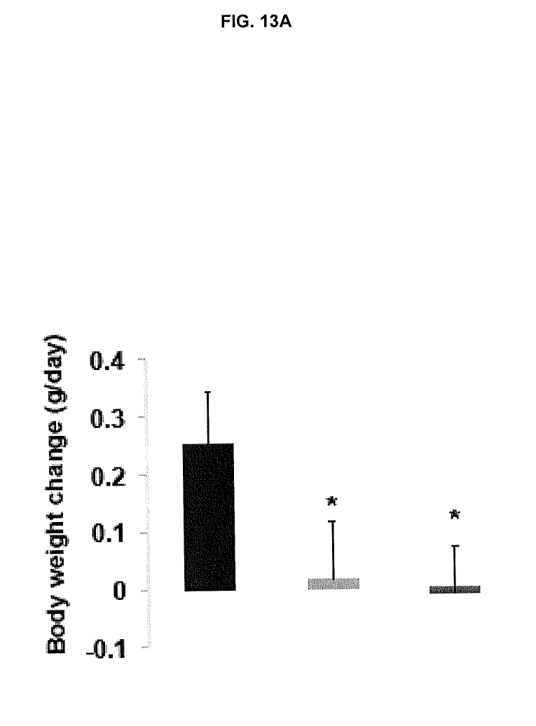

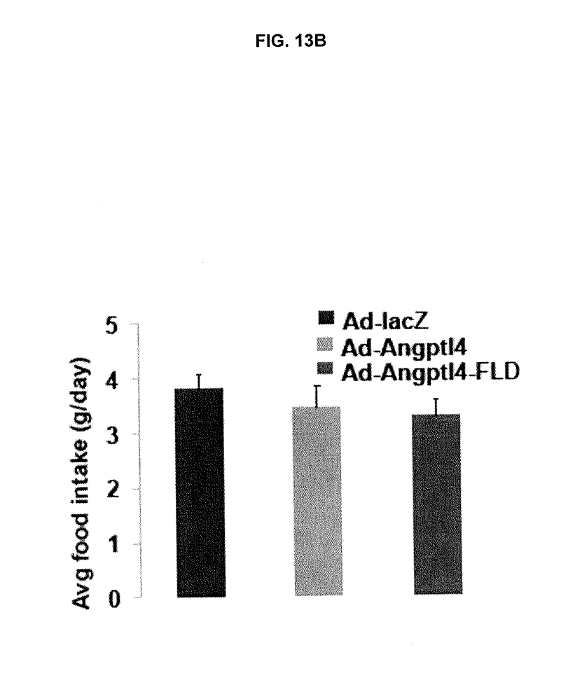

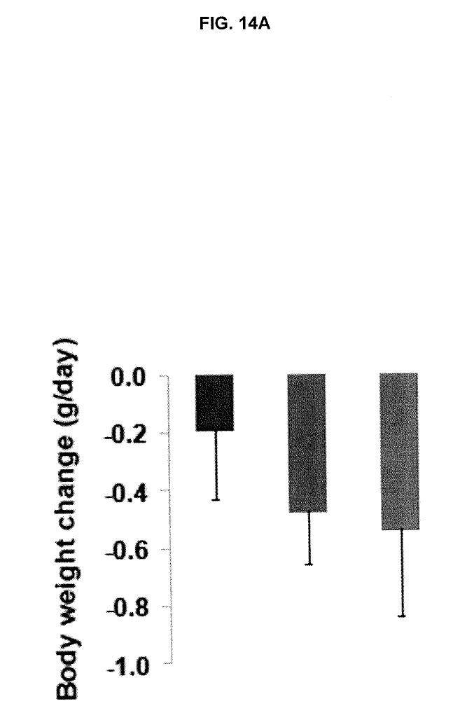

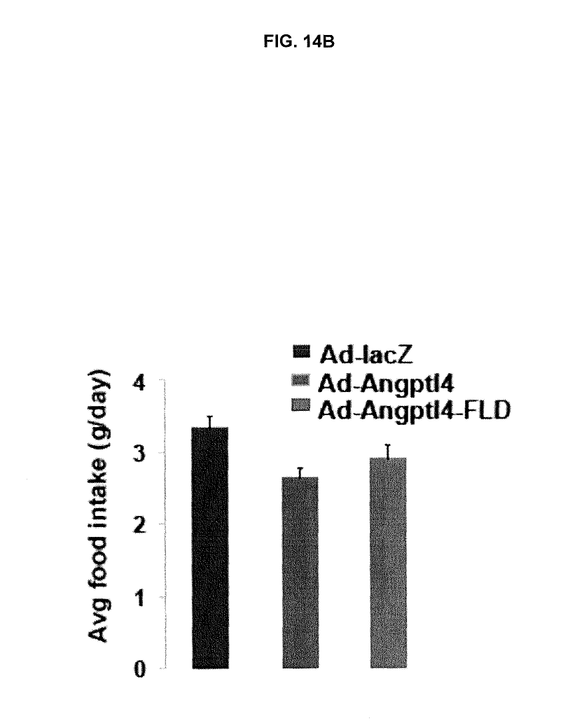

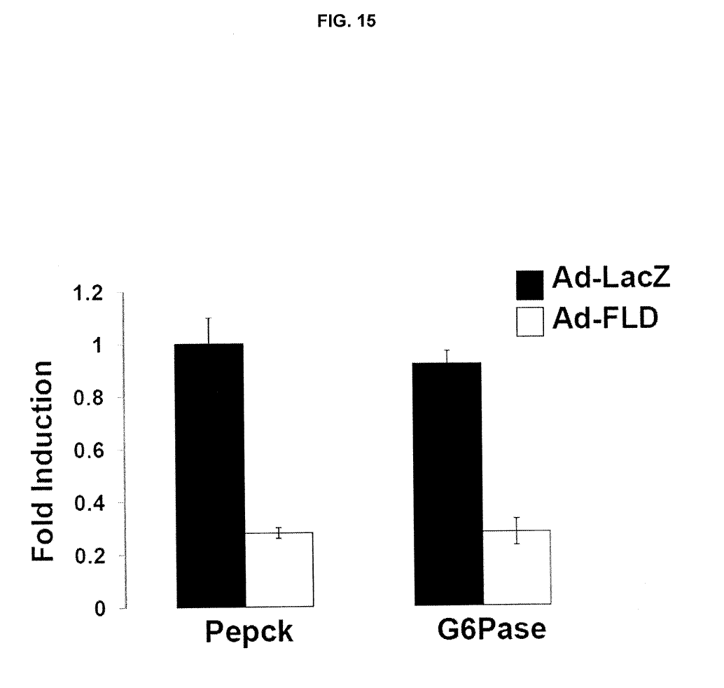

We next performed GTT in these LacZ-, FLD- and ANGPTL4-overexpressing chow diet fed mice. As shown in FIG. 11, FLD- and ANGPTL4-overexpressing mice were more glucose tolerant than LacZ-overexpressing mice. Because TG levels were not affected by ANGPTL4 and FLD overexpression, the improved glucose tolerance was unlikely due to the decreased adiposity. We thus test whether gluconeogenesis was reduced in FLD-overexpressing mice. We found that FLD- and ANGPTL4-overexpressing mice indeed had improved pyruvate tolerance (FIG. 12A). We compared the expression of Pepck gene in liver of FLD-, ANGPTL4- and LacZ-overexpressing mice. We found that the expression of Pepck and G6Pase was approximately 70% lower in FLD-overexpressing mice than that of LacZ-overexpressing mice (FIG. 12B). To learn whether FLD effect on Pepck gene expression was a cell autonomous effect we infected H4IIE rat hepatoma cells with adenovirus expressing LacZ or FLD for 3 days. At the 3rd day, we treated mice with ethanol (EtOH, vehicle control) or Dex for 5 hr. Gene expression analysis showed that overexpression of FLD reduced Dex-induced Pepck and G6Pase gene expression. We also treated mouse primary hepatocytes directly with Dex or FLD plus Dex for 5 hr. In this case, FLD suppressed Dex-induced Pepck gene expression for approximately 40%. Overall, these results suggest that the inhibition of Pepck and G6Pase gene expression by FLD is a cell autonomous effect.

### Example 5

### Amino Acid Sequences of FLD Used in Our Studies:

Amino acid 38-165 from wild type ANGPTL4 was deleted, and 3 copies of FLAG-Tagged sequences (final 24 amino acids) were added. FLAG-Tagged sequences are used to purify this protein.

msgaptagaa lmlcaatavl lsaqggpvqs ksprfas pemaq
pvdpahnvsr lhrlprdcqe lfqvgerqsg lfeiqpqgsp
pflvnckmts dggwtviqrr hdgsvdfnrp weaykagfgd
phgefwlgle kvhsitgdrn srlavqlrdw dgnaellqfs
vhlggedtay slqltapvag qlgattvpps glsvpfstwd
qdhdlrrdkn cakslsggww fgtcshsnln gqyfrsipqq
rqklkkgifw ktwrgryypl qattmliqpm aaeaas DYKDDDDK
DYKDDDDK DYKDDDDK.

First 37 amino acids contain signal peptide that allows FLD to be secreted to outside of cells. If we are going to inject FLD protein, these 37 amino acids are not needed. Also FLAG-Tagged (final 24 amino acids) may not needed, which depends on the method we choose to create FLD.

### Example 6

Adipocytes play a central role in the regulation of energy homeostasis. White adipose tissue (WAT) store excess energy whereas brown adipose tissue (BAT) dissipates energy in the form of heat to defend cold and obesity. Prolonged exposure of norepinephrine or other agents that can increase cyclic AMP (cAMP) levels in adipocytes have been shown to induce “browning” of WAT, a process that certain white adipocytes in WAT develop the phenotypes of brown adipocytes. Moreover, cAMP-Protein kinase A (PKA) signaling can also stimulate the expression of uncoupling protein 1 (Ucpl) gene that encodes a protein that can generate heat in BAT. PKA signaling also induces lipolysis, which generates fatty acids that are required for Ucpl activation. Because FLD can promote cAMP-PKA signaling in adipocytes, we examine whether increasing FLD levels in plasma augments energy expenditure in high fat diet fed mice. Energy expenditure can be estimated based on oxygen consumption. Assuming glucose is the only source for energy, every glucose molecule will be converted to carbon dioxide and water. In this process, exactly 6 oxygen molecules are used. Thus, if only glucose is being used for energy, we could precisely convert oxygen consumption to glucose burnt and thus to energy expenditure.

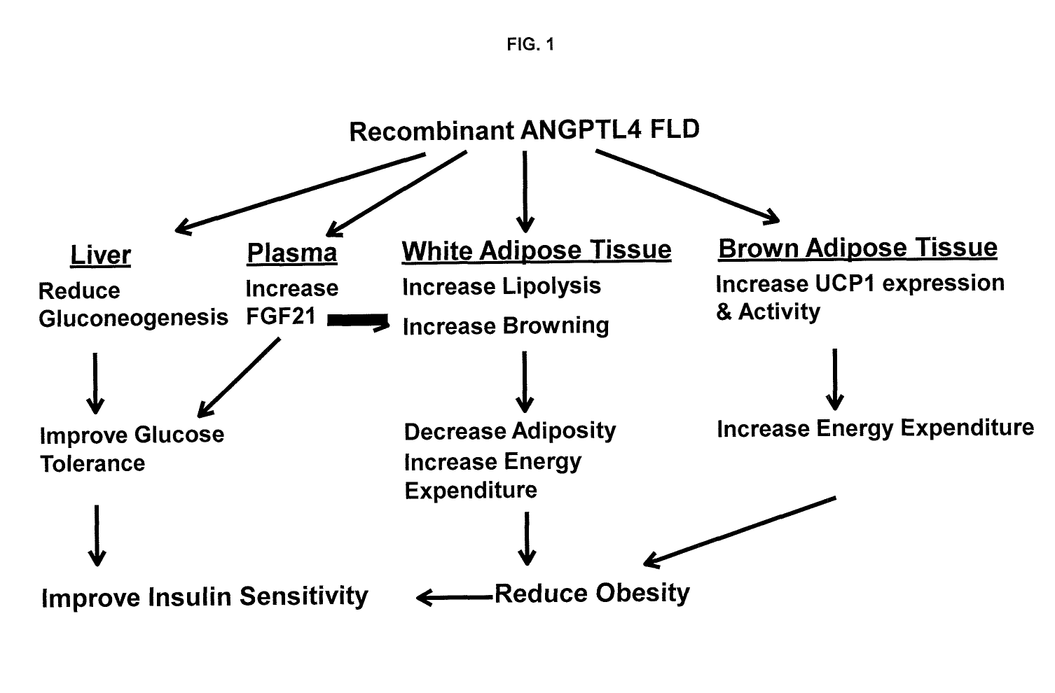

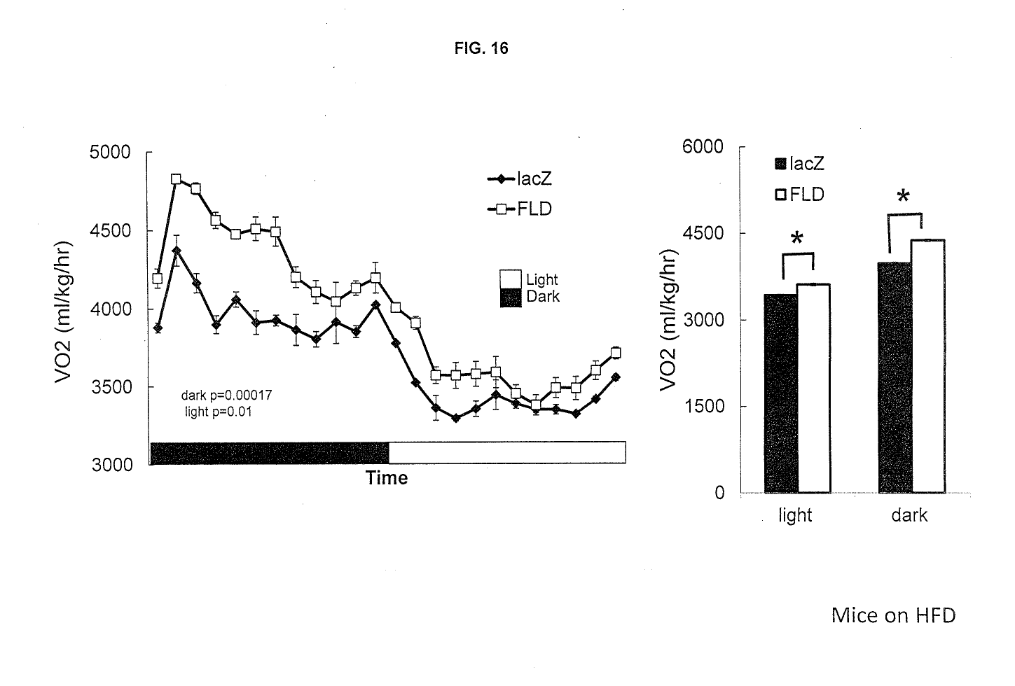

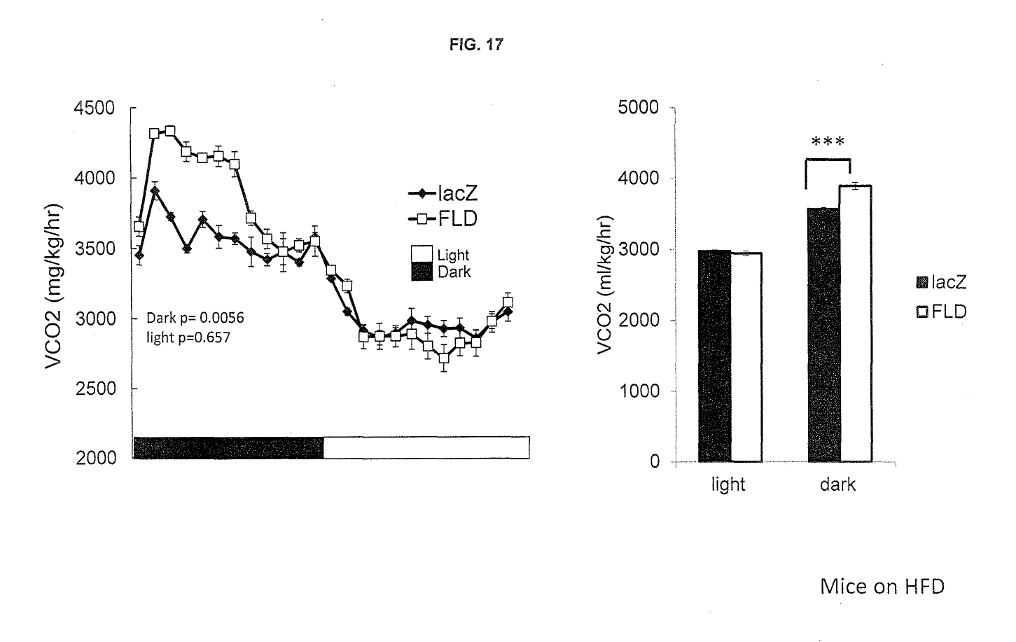

Mice were infected with adenovirus expressing FLD or LacZ (control) and placed under high fat diet. After 3 weeks, they were placed in an environment-controlled Comprehensive Laboratory Animal Monitoring System (CLAMS) with cages equipped with an indirect open-circuit calorimeter (Oxymax Equal Flow System) to measure oxygen consumption (V_{O2}) and carbon dioxide production (V_{CO2}). We found that both V_{O2} and V_{CO2} were higher during dark cycle in mice overexpressing FLD than those overexpressing LacZ (FIG. 16 and FIG. 17). In light cycle, mice overexpressing FLD also had higher V_{O2} (FIG. 1). These results indicated that increasing FLD in plasma elevates energy expenditure in high fat diet fed mice.

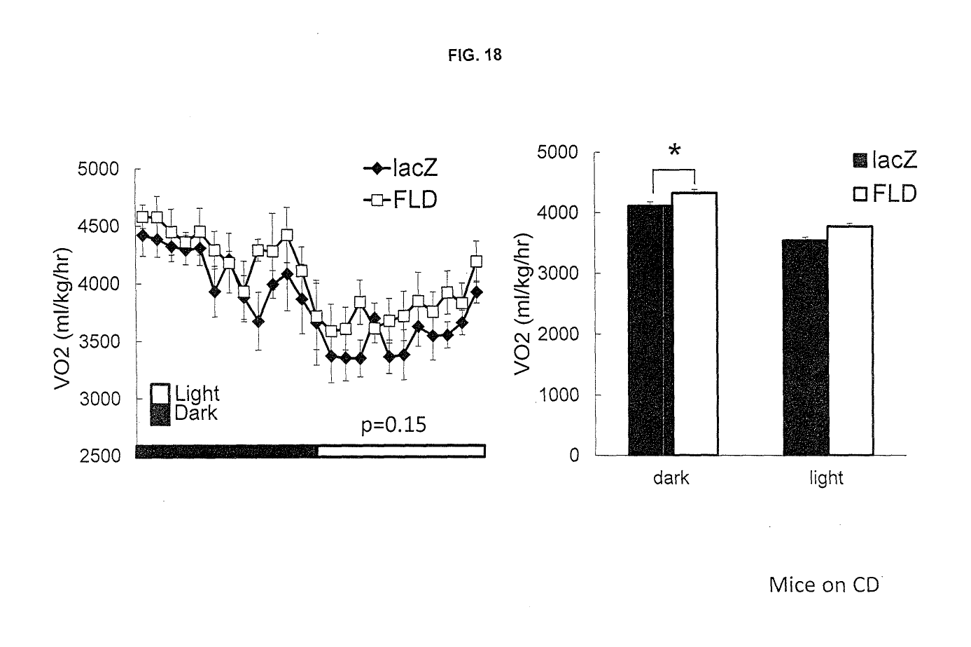

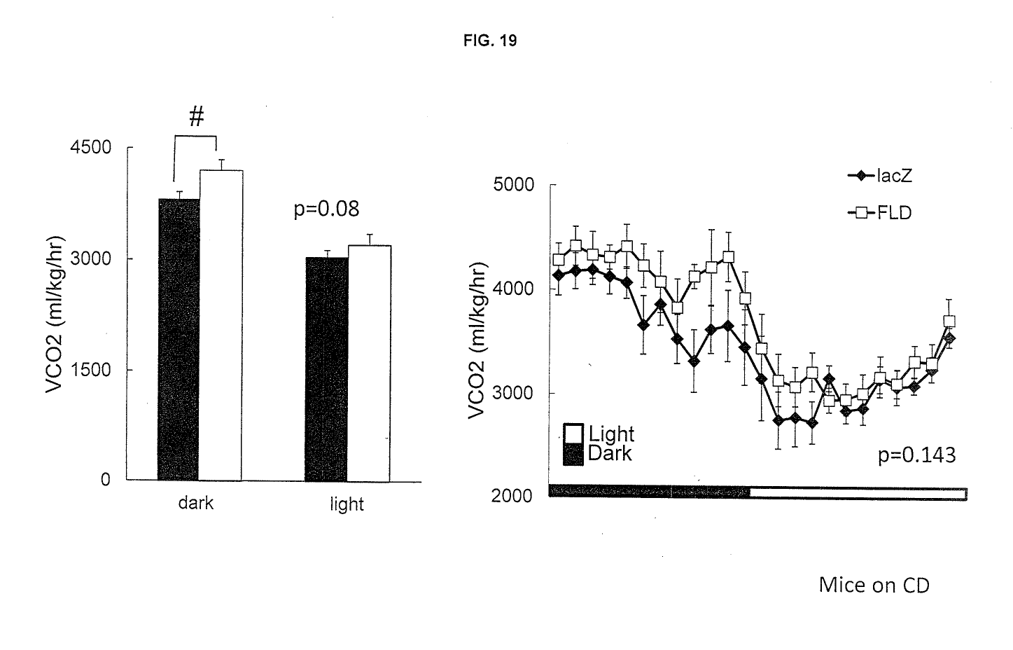

We also tested whether elevating plasma FLD levels in chow diet fed mice modulate their energy expenditure. Mice were infected with adenovirus expressing FLD or LacZ (control) and placed under chow diet. After 3 weeks, these mice were placed in CLAMS with cages equipped with an indirect open-circuit calorimeter to measure V_{O2} and V_{CO2}. We found that V_{O2} were higher in both light and dark cycle in mice overexpressing FLD than those overexpressing LacZ (FIG. 18). Mice overexpressing FLD also had higher V_{CO2} in light cycle (FIG. 19), whereas during dark cycle V_{CO2} levels were trending higher (p=0.08, FIG. 19). These results indicated that increasing FLD in plasma also elevates energy expenditure in chow diet fed mice.

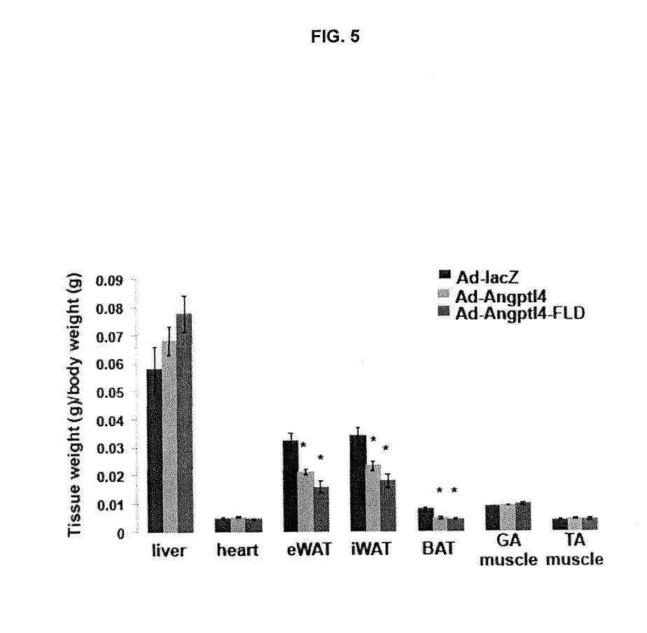

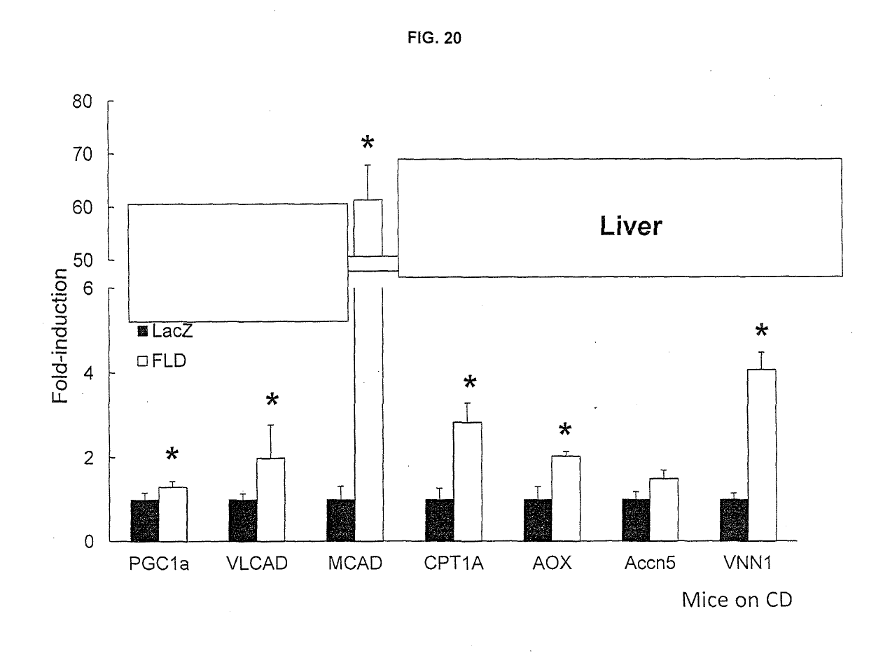

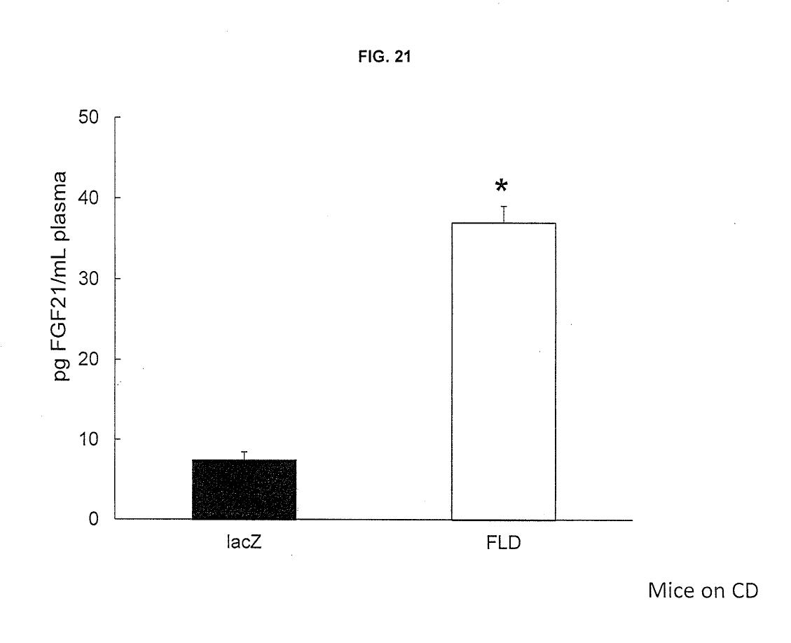

Increasing FLD in plasma elevates the expression of fatty acid oxidation genes in liver of high fat diet fed mice. Whether increasing plasma FLD levels in chow diet fed mice also elevates fatty acid oxidation genes in liver of chow diet fed mice was tested. It was found that these fatty acid oxidation genes, including Vlcad, Mcad, Cptla, Pgc-lα and Aox1, were all augmented after three weeks of FLD overexpression (FIG. 5). Vnn1 is a peroxisome proliferator activated receptor α (PPARα) target gene, whereas Accn5 is activated by PPARδ/β. The Vnn1 but not Accn5 was induced by FLD overexpression (FIG. 20). These results suggested PPARα was activated by FLD overexpression in liver. Fibroblast growth factor 21 (FGF21) is a PPARα target gene in liver, which encodes a hormone that can increase energy expenditure by promoting browning of WAT. Plasma FGF21 levels were higher in FLD overexpressing mice than those of LacZ overexpressing mice (FIG. 21). These results suggested that the alternative way for FLD to increase energy expenditure is to augment FGF21 production.

FLD appears to increase energy expenditure to reduce adiposity. The increase of FGF21 plasma levels is likely involved in such FLD effect (FIG. 1).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A peptide comprising the amino acid sequence of SEQ ID NO: 2, or a phallnaceutically acceptable salt thereof.

2. A peptide comprising a sequence having at least about 95% sequence identity with amino acids165-406 of SEQ. ID. NO.: 1.

3. A peptide according to claim 5, comprising a sequence having at least about 95% identity with amino acids 184-406 of SEQ. ID. NO.: 1.

4. A conjugate comprising the peptide of claim and a heterologous moiety covalently bound thereto.

5. A dimer or multimer comprising the peptide, or a conjugate thereof, of claim 1 and a heterologous peptide or polypeptide.

6. A pharmaceutical composition comprising the peptide, or a conjugate thereof, of claim 1 and a pharmaceutically acceptable carrier.

7. A method of lowering body weight in a subject in need thereof, said method comprising administering to the subject an effective amount of the peptide of claim 1.

8. A method of reducing blood glucose levels in a subject in need thereof, said method comprising administering to the subject an effective amount of the peptide of claim 1.

9. A method of treating diabetes in a subject in need thereof, said method comprising administering to the subject an effective amount of the pharmaceutical composition of claim 4.

10. A method of treating obesity in a subject in need thereof, comprising administering to the subject an effective amount of the peptide of claim 1.

11. A method of lowering body weight in a subject in need thereof, comprising administering to the subject an effective amount of the pharmaceutical composition of claim 6.

12. A method of reducing blood glucose levels in a subject in need thereof, comprising administering to the subject an effective amount of the pharmaceutical composition of claim 6.

13. A method of treating diabetes in a subject in need thereof, comprising administering to the subject an effective amount of the pharmaceutical composition of claim 6.

14. A method of treating obesity in a subject in need thereof, comprising administering to the subject an effective amount of the pharmaceutical composition of claim 6.
